(19) United States
(12) Patent Application Publication
(10) Pub. No.: US 2017/0333752 A1
(43) Pub. Date: Nov. 23, 2017

(54) ENHANCING MONITORING OF SWIMMING

(71) Applicant: Polar Electro Oy, Kempele (FI)

(72) Inventors: Seppo Korkala, Kempele (FI); Marko Tilvis, Oulunsalo (FI); Ville Majava, Kiviniemi (FI); Harri Määttä, Kempele (FI); Jonna Puurunen, Oulu (FI); Ville Kampman, Oulu (FI); Lauri Lumme, Oulu (FI)

(21) Appl. No.: 15/593,522

(22) Filed: May 12, 2017

(30) Foreign Application Priority Data
May 19, 2016 (GB) 1608799.1

(51) Int. Cl. A63B 24/00; A61B 5/0205; A63B 33/00; A63B 71/06; A61B 5/00; A61B 5/11; A61B 5/024

(52) U.S. Cl. CPC A63B 24/0062; A63B 71/0686; A61B 5/0205; A61B 5/6803; A63B 33/002; A63B 71/0622; A61B 5/0015; A63B 2207/02; A63B 2244/20; A63B 2071/0625; A63B 2033/004; A61B 5/02416; A61B 5/1118; A61B 5/02438; A61B 2560/0214; A61B 2503/10; A63B 2208/03; A63B 2220/30; A63B 2220/836; A63B 2225/50; A63B 2230/065; A63B 2071/0666

(57) ABSTRACT

A device for measuring physical activity of a user during swimming is provided, which includes the following: a sensor unit adapted and dimensioned to be coupled with swimming goggles, wherein the sensor unit includes a motion circuitry configured to measure physical motion of the user; an optical cardiac activity circuitry configured to be placed at least partially against a body tissue of the user and to measure cardiac activity of the user; and a wireless communication circuitry configured to transfer data between the device and at least one external device, wherein the wireless communication circuitry is configured to transfer physical activity data of the user to the at least one external device, and the physical activity data includes at least one of motion data obtained using the motion circuitry, cardiac activity data obtained using the cardiac activity circuitry.

### CROSS-REFERENCE TO RELATED APPLICATION

This application claims benefit to United Kingdom Application No. 1608799.1, filed May 19, 2016, which is incorporated by reference herein in its entirety.

### BACKGROUND

### Field

This invention relates to monitoring physical activity of a user. More particularly, the invention relates to monitoring the physical activity of a user during swimming.

### Description of the Related Art

Monitoring physical exercise is becoming increasingly popular. Physical exercises, such as running, cycling, and swimming, may each bring forth different environments which may require different kind of features from the monitoring devices and systems. Therefore, it may be beneficial to introduce solutions that enhance monitoring physical activity of a user during swimming.

### SUMMARY

According to an aspect, there is provided a device for measuring physical activity of a user during swimming, the device comprising: a sensor unit adapted and dimensioned to be coupled with swimming goggles, the sensor unit comprising a motion circuitry configured to measure physical motion of the user, an optical cardiac activity circuitry configured to be placed at least partially against a body tissue of the user and to measure cardiac activity of the user, and a wireless communication circuitry configured to transfer data between the device and at least one external device, wherein the wireless communication circuitry is configured to transfer physical activity data of the user to the at least one external device, the physical activity data comprising at least one of motion data obtained using the motion circuitry, cardiac activity data obtained using the cardiac activity circuitry.

In an embodiment, the wireless communication circuitry comprises a Bluetooth circuitry.

In an embodiment, the wireless communication circuitry is configured to operate on a 5 kHz radio band.

In an embodiment, the sensor unit is configured to be coupled with an audio module comprising an electroacoustic transducer configured to be placed against the body tissue of the user and to convert electrical signals into mechanical vibrations to produce sound and/or to detect sound as mechanical vibrations and to covert the mechanical vibrations into electrical signals.

In an embodiment, the electroacoustic transducer comprises a bone conductor transducer.

In an embodiment, the device comprises the audio module.

In an embodiment, the device is configured to cause the audio module to output one or more sound notifications.

In an embodiment, device is configured to cause the audio module to output at least one of a notification indicating a heart rate zone of the user, a notification indicating a heart rate of the user.

In an embodiment, the device is configured to detect a turn by the user during swimming and to cause the audio module to output a notification indicating a recorded time.

In an embodiment, the recorded time comprises at least one of a lap time of the user, a pool length time of the user, a total swimming time.

In an embodiment, the device is further configured to determine a glide phase of the turn and to cause the audio module to output the notification indicating the recorded time during the glide phase.

In an embodiment, the detecting the turn is based on at least one of the motion data obtained using the motion circuitry, data obtained using one or more external sensor devices worn by the user.

In an embodiment, the sensor unit is detachably attachable to the audio module.

In an embodiment, the audio module comprises at least one attachment element for attaching the device to a strap of the swimming goggles.

In an embodiment, the device comprises an interface element configured to enable electrical coupling of the sensor unit with the audio module.

In an embodiment, the interface element is configured to enable the sensor unit to be communicatively coupled with the audio module.

In an embodiment, the sensor unit comprises a power source, and wherein the interface element and the power source are configured to provide operational voltage to the audio module.

In an embodiment, the operational voltage to the audio module is transferred via the interface element as alternating current voltage.

In an embodiment, the sensor unit is configured to operate in a control mode when the sensor unit is electrically coupled with the audio module, and wherein in the control mode the sensor unit is further configured to control the audio module to output sound.

In an embodiment, the sensor unit is configured to operate in a maintenance mode when the sensor unit is electrically coupled with an external device, and wherein in the maintenance mode the sensor unit is further configured to be recharged by said external device, to transmit physical activity data to said external device, and/or to receive configuration data from said external device.

In an embodiment, the interface element comprises a first interface element in the sensor unit and a second interface element in an audio module of the device, the first and second interface elements configured to be electrically coupled to each other and to enable at least one of communication between the sensor unit and the audio module, and transfer of operational voltage from the sensor unit to the audio module.

In an embodiment, the device is configured to determine, based on the motion data and/or measurement by the wireless communication circuitry, at least one time period when the device is above water during swimming, and wherein the device is further configured to transfer the physical activity data of the user to the at least one external device during said at least one time period.

In an embodiment, the device is configured to determine that the device is used during swimming, and wherein the device is further configured to decrease transmission interval of the wireless communication circuitry during swimming.

In an embodiment, the device further comprises: an electromagnetic flowmeter configured to measure swimming speed of the user, wherein the physical activity data comprises the swimming speed and/or the device is further configured to cause the audio module to output a notification indicating the swimming speed.

In an embodiment, the device further comprises: one or more light emitting diodes configured to output visual notifications to the user.

In an embodiment, the device further comprises: one or more light guides configured to convey light emitted by the one or more light emitting diodes to one or more lens areas of the swimming goggles.

According to an aspect, there is provided a device for measuring physical activity of a user during swimming, the device comprising: a sensor unit adapted and dimensioned to be coupled with a swimming goggles, the sensor unit comprising a motion circuitry configured to measure physical motion of the user, an optical cardiac activity circuitry configured to be placed at least partially against a body tissue of the user and to measure cardiac activity of the user, and a wireless communication circuitry configured to transfer data between the device and at least one external device, wherein the wireless communication circuitry is configured to transfer physical activity data of the user to the at least one external device, the physical activity data comprising at least one of motion data obtained using the motion circuitry, cardiac activity data obtained using the cardiac activity circuitry, the sensor unit comprising: an interface element and a controller, the controller configured to change an operating mode of the sensor unit such that when the interface element is electrically coupled with a corresponding interface element of an audio module, the sensor unit operates in a control mode in which the sensor unit controls the audio module to output sound, and such that when the interface element is electrically coupled with a corresponding interface element of an external device providing a power input, the sensor unit operates in a maintenance mode in which the sensor unit is recharged by said external device providing said power input; wherein the audio module comprises an electroacoustic transducer configured to be placed against the body tissue of the user and to convert electrical signals into mechanical vibrations to produce sound and/or to detect sound as mechanical vibrations and to covert the mechanical vibrations into electrical signals; wherein the device is configured to cause the audio module to output one or more sound notifications; wherein the device is configured to detect a turn by the user during swimming and to cause the audio module to output a notification indicating a recorded time; and wherein the device is further configured to determine a glide phase of the turn and to cause the audio module to output a notification indicating the recorded time during the glide phase.

Some embodiments are defined in the dependent claims.

One or more examples of implementations are set forth in more detail in the accompanying drawings and the description below. Other features will be apparent from the description and drawings, and from the claims.

### BRIEF DESCRIPTION OF DRAWINGS

In the following the invention will be described in greater detail by means of preferred embodiments with reference to the attached drawings, in which

FIG. 1 illustrates an example system to which the embodiments may be applied;

FIGS. 2A to 2C illustrate some embodiments;

FIGS. 3A to 3C illustrate some embodiments;

FIG. 4 illustrates an embodiment;

FIGS. 5A to 5C illustrate some embodiments;

FIGS. 6A to 6B illustrate some embodiments;

FIGS. 7A to 7C illustrate some embodiments;

FIGS. 8A to 8C illustrate some embodiments;

FIGS. 9A to 9B illustrate some embodiments;

FIG. 10 illustrates an embodiment; and

FIG. 11 illustrates an embodiment.

### DETAILED DESCRIPTION

The following embodiments are exemplifying. Although the specification may refer to “an”, “one”, or “some” embodiment(s) in several locations of the text, this does not necessarily mean that each reference is made to the same embodiment(s), or that a particular feature only applies to a single embodiment. Single features of different embodiments may also be combined to provide other embodiments. Furthermore, words “comprising” and “including” should be understood as not limiting the described embodiments to consist of only those features that have been mentioned and such embodiments may contain also features/structures that have not been specifically mentioned.

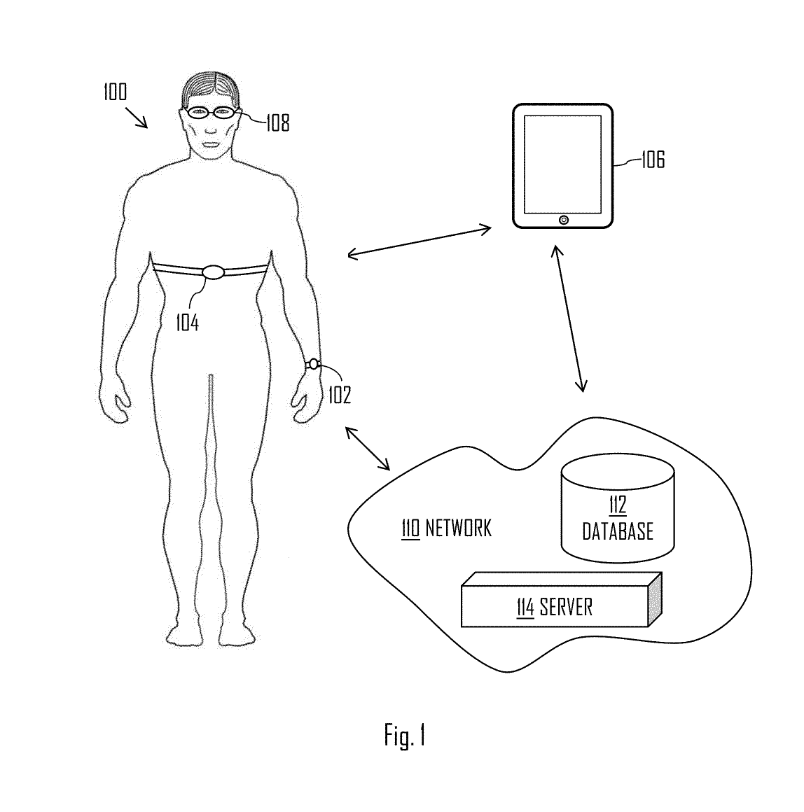

FIG. 1 illustrates an example system to which the embodiments of the invention may be applied. Referring to FIG. 1, a user 100 wearing one or more devices may be shown. The devices may comprise a wrist unit 102, one or more external sensor devices 104, and/or swimming goggles 108. Further, the system of FIG. 1 may comprise a portable electronic device 106 (e.g. used by the user 100) and/or network 110 (e.g. a training network) comprising at least one database 112 and at least one server 114.

The wrist unit 102 may comprise one or more sensors configured to measure physical activity of the user 100. Examples of these sensors may comprise accelerometer(s), gyroscope(s), satellite positioning circuitry (e.g. a global navigation satellite system (GNSS) circuitry, such as a Global Positioning System (GPS) and/or a GLObal NAvigation Satellite System (GLONASS)), and/or cardiac activity circuitry (e.g. optical cardiac activity circuitry). The wrist unit 102 may be used to monitor physical activity of the user 100 by the user 100, for example. Thus, the wrist unit 102 may comprise a display and user interface. Further, the wrist unit 102 may comprise communication circuitry enabling wireless and/or wired data communication with other device(s) of the system.

The one or more external sensor devices 104 may comprise sensor(s) worn by the user 100 and/or sensor which may be used by the user 100. For example, a cardiac activity belt may be worn by the user 100 as shown in FIG. 1. Another example may be a weight sensor (e.g. a scale) which may be occasionally used by the user 100. Other examples may comprise bike sensor(s), motion sensor(s), stride sensor(s), cadence sensor (s) and temperature sensor(s), to name a few examples. The cadence sensor(s) may, for example, be worn on a hand or a leg of the user to measure swimming cadence (e.g. strokes or kicks per minute). The external sensor device(s) 104 may comprise sensors, such as a heart rate transmitter, heart rate sensor, a stride sensor, a positioning sensor, a bioimpedance sensor, a cadence sensor, and a power sensor, to mention a few. The external sensor device(s) 104 may transmit the sensor data to an external device, such as the wrist unit 102, to some other wearable device, to the portable electronic device 106 and/or to a server 114. The server 114 may be accessible via a network 110. The wrist unit 102, the portable electronic device 106 and/or the server 114 may receive said sensor data. Thus, the external sensor devices(s) 104 may comprise wireless and/or wired communication capabilities. Further, the external sensor device(s) 104 may comprise one or more memories for storing data, such as the sensor data. During swimming, for example, it may be beneficial to load the sensor data to an external device after the swimming session is over due to possible data connection problems caused by water.

The portable electronic device 106, such as mobile phone, tablet computer, laptop or similar device, may also be used to monitor physical activity of the user 100. For example, in some implementations, the wrist unit 102 may not be necessary as the sensor device(s) 104 may transmit data directly to the server 114 and/or to the portable electronic device 106. Thus, the portable electronic device 106 may be used to view or monitor data concerning the physical activity (e.g. from memory of the portable electronic device 106) or via the network 110 (e.g. data stored in the database 112). Also in some implementations, the wrist unit 102 may transmit data to the portable electronic device 106 and/or the server 114.

The system described in relation to FIG. 1 may be suitable for monitoring physical activity during different kinds of sports. However, one aspect that is characteristics to swimming, are the swimming goggles 108. Anybody who swims regularly, acknowledges the need to protect his/her eyes from water.

Therefore, swimming goggles are quite regularly used by swimmers. However, swimming goggles are not conventionally utilized for enhancing monitoring of swimming although they provide a platform for implementing different sensor structures for measuring physical activity during swimming. Therefore, there is provided a wearable device for measuring physical activity of a user during swimming. The wearable device may be configured to be coupled with the swimming goggles 108. Thus, when the user 100 is wearing his/her swimming goggles 108, the wearable device may be used to monitor the swimming exercise. Thus, for example, a need to use further sensor device(s) (e.g. a heart rate belt) may be reduced, and thus the monitoring of swimming may become even more effortless. Further, in some cases, it may be beneficial to use more than one measurement capable elements to obtain, for example, more reliable results. For example, two or more sensors (e.g. one in swimming glasses and one in the wrist unit 102) may be utilized. For example, one motion sensor may be comprised in the wearable device and another sensor may be comprised in the wrist unit 102.

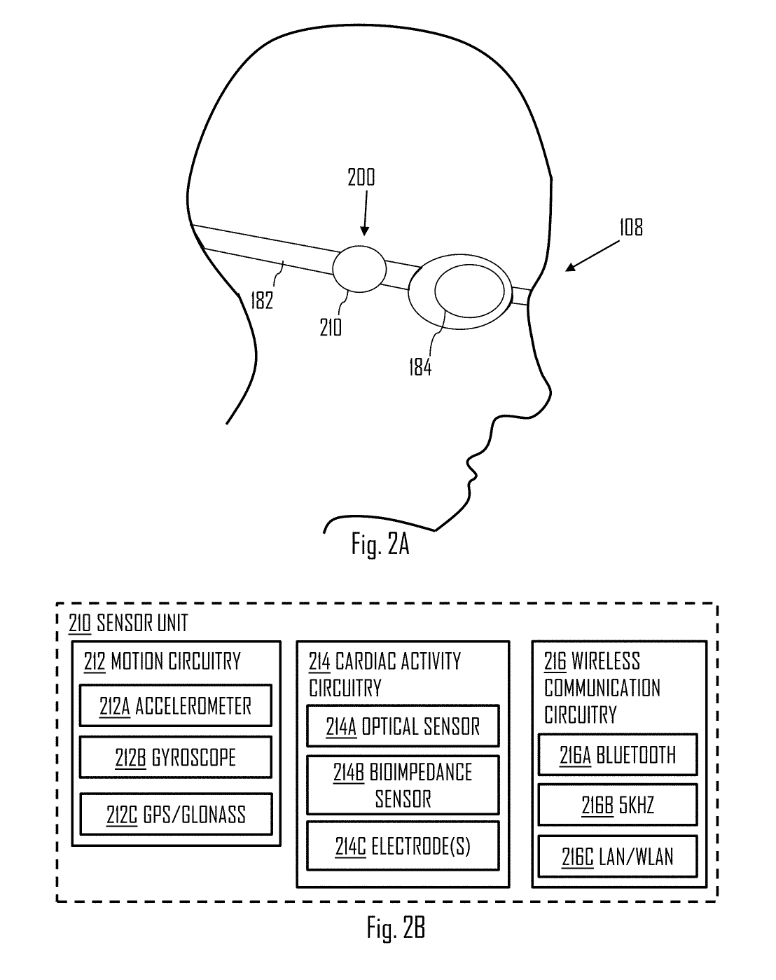

FIGS. 2A to 2C illustrate some embodiments. Referring to FIG. 2A, a device 200 for measuring physical activity of a user during swimming may be shown. The device 200 may comprise a sensor unit 210. The sensor unit 210 may be adapted and dimension to be coupled with the swimming goggles 108. In more general terms, the device 200 may be adapted and dimension to be coupled (e.g. physically coupled) with the swimming goggles 108. The swimming goggles 108 may be any kind of goggles that are suitable to be used during swimming. The swimming goggles 108 may comprise one or more lenses 184 (only one shown in FIG. 2A, but there may be a similar lens for the other eye) and an attachment element 182 (e.g. strap) for attaching the swimming goggles 108 to the user.

Referring to FIG. 2B, the sensor unit 210 may comprise a motion circuitry 212, a cardiac activity circuitry 214, and/or a wireless communication circuitry 216. In an embodiment, the sensor unit 210 comprises a motion circuitry 212 configured to measure physical motion of the user, an optical cardiac activity circuitry 214A configured to be placed at least partially against a body tissue of the user and to measure cardiac activity of the user, and a wireless communication circuitry 216 configured to transfer data between the device 200 and at least one external device, wherein the wireless communication circuitry 216 is configured to transfer physical activity data of the user to the at least one external device, the physical activity data comprising at least one of motion data obtained using the motion circuitry 212, cardiac activity data obtained using the optical cardiac activity circuitry 214A.

The motion circuitry 212 may be configured to measure physical motion data of the user. Physical motion data may comprise velocity data, acceleration data, direction data, orientation data, and/or position data, for example. The motion circuitry 212 may comprise one or more accelerometers 212A, one or more gyroscopes 212B, and/or one or more satellite positioning circuitries 212C (e.g. GPS and/or GLONASS circuitries). In some embodiments, the motion circuitry 212 (also referred to as motion sensing circuitry 212) comprises one or more magnetometers for measuring the direction and/or orientation data.

In an embodiment, the motion circuitry 212 comprises an accelerometer and a gyroscope. The motion circuitry 212 may further comprise sensor fusion software for combining the accelerometer data and gyroscope data so as to provide physical quantities, such as acceleration data, velocity data, or limb trajectory data in a reference coordinate system having orientation defined by a predetermined gyroscope orientation.

In an embodiment, the motion circuitry 212 comprises a gyroscope and a magnetometer. The motion circuitry 212 may further comprise sensor fusion software to combine gyroscope data and magnetometer data so as to provide a reference coordinate system for the gyroscope based on the Earth magnetic field measured by the magnetometer. In general, the sensor fusion software described above may combine measurement data acquired from at least two motion sensors such that measurement data acquired from one motion sensor is used to establish the reference coordinate system for the measurement data acquired from at least one other motion sensor. Thus for example, the satellite positioning data may also be utilized in the sensor fusion.

In an embodiment, the sensor unit 210 comprises a sensor fusion circuitry configured to combine data from two or more sensors. The two or more sensors may be comprised in the sensor unit 210 and/or in some external device(s) (e.g. external sensor device(s) 104). For example, the sensor fusion circuitry may be configured to combine data from one or more accelerometers, one or more gyroscopes, and/or one or more satellite positioning circuitries.

In an embodiment, the sensor fusion circuitry is configured to combine data from two or more sensors of the motion circuitry 212. Thus, the motion data may be combination of data obtained using one or more motion sensors. Similar logic may in some embodiments apply to cardiac activity data also. This may make the motion and/or cardiac activity data more reliable and accurate.

In an embodiment, the sensor fusion circuitry is configured to combine data from two or more motion sensors into the motion data. One or more motion sensors may be comprised in the sensor unit 210, whereas at least one other motion sensor may be comprised in the external sensor device(s) 104. This may provide more accurate motion data.

The cardiac activity circuitry 214 may be configured to be placed at least partially against a body tissue of the user and to measure cardiac activity data of the user. The cardiac activity circuitry 214 may comprise one or more optical sensors 214A, one or more bioimpedance sensors 214B, and/or one or more electrodes (e.g. ECG measurement). Sensor fusion may, in some embodiments, be applied to the cardiac activity circuitry 214. Cardiac activity data may comprise, for example, heart rate of the user, heart rate zone(s) of the user, Heart Beat Interval (HBI) of the user and/or Heart Rate Variability (HRV) of the user. It may be beneficial to use optical measurement (e.g. the one or more optical sensors 214A, also referred to as optical cardiac activity circuitry) to measure HBI and/or HRV as head area may provide good position for such measurement. The bioimpedance sensor(s) 214B may be configured to measure cardiac activity of the user. Also, the bioimpedance sensor(s) may configured to, for example, measure skin conductivity and/or skin temperature of the user. This conductivity and/or temperature information may be comprised in the transmitted physical activity data, for example.

Still referring to FIG. 2B, the wireless communication circuitry 216 may be configured to transmit data from the device 200 and/or the sensor unit 210 to one or more external devices. Similarly, data may be transmitted to the device 200 and/or the sensor unit 210 using the wireless communication circuitry 216. In an embodiment, the sensor unit 210 comprises a communication circuitry configured to enable wired (e.g. Universal Serial Bus (USB)) connection to one or more external devices. The sensor unit 210 and/or the device 200 may comprise one or more antennas to enable the communication circuitry 216 to transfer electromagnetic energy via air-interface. That is, the wireless communication circuitry 216 may transform the physical activity data into electromagnetic energy that is transmitted via the one or more antennas to an external device.

In an embodiment, the wireless communication circuitry 216 comprises a Bluetooth circuitry 216A. The Bluetooth circuitry 216A may enable data transfer and/or communication according to the Bluetooth specifications. For example, the wireless communication circuitry 216 may support Bluetooth Light Energy (BLE) (also referred to as Bluetooth smart).

In some embodiments, the wireless communication circuitry 216 supports Near Field Communication (NFC) and/or similar induction based proximity communication technologies. In an embodiment, the wireless communication circuitry 216 supports induction based proximity communication.

In an embodiment, the wireless communication circuitry 216 supports ANT, ANT+, and/or ZigBee communications. Any suitable RF technique may be applied.

In an embodiment, the wireless communication circuitry 216 is configured to operate on a 5 kHz band (i.e. 5 kilohertz band). In an embodiment, the wireless communication circuitry 216 comprises a circuitry 216B supporting communication on the 5 kHz band. 5 kHz may be beneficial to be used in water environment compared to some other communication technologies. The wavelength of signals transmitted on the 5 kHz band may be quite long, and thus may better penetrate water. Therefore, data transfer may be more reliable using 5 kHz band at least in some embodiments.

In an embodiment, the wireless communication circuitry 216 comprises a Local Area Network (LAN) and/or wireless LAN (WLAN) circuitry 216C (e.g. WiFi). Thus, for example, the device 200 and/or the sensor unit 210 may transfer data with an external device via WLAN communication. WLAN circuitry 216C may support direct connection between two devices. Thus, for example, the sensor unit 210 may be directly connected to another device that also supports such connection.

In an embodiment, the sensor unit 210 comprises a memory configured to store the physical activity data (e.g. motion data, cardiac activity data) during swimming. The sensor unit 210 may thus store the physical activity data during swimming, and transmit said data after the swimming has ended.

In an embodiment, the wireless communication circuitry 216 is configured to transmit and/or receive data during swimming. Thus, the physical activity data may be transmitted during swimming, for example.

Referring to FIG. 2C, according to an aspect, there is provided an audio module 220 comprising an electroacoustic transducer configured to be placed against the body tissue of the user and to convert electrical signals into mechanical vibrations to produce sound and/or to detect sound as mechanical vibrations and to covert the mechanical vibrations into electrical signals.

In an embodiment, the device 200 comprises the audio module 220.

As discussed, the audio module 220 may take sound as an input (e.g. input electroacoustic transducer 222, such as a microphone) and/or output sound (e.g. output electroacoustic transducer 224, such as a speaker). It needs to be noted that the audio module 220 may comprise one or more electroacoustic transducers 222, 224 (e.g. one or more transducers for input and/or one or more transducers for output). However, one electroacoustic transducer may, in some embodiments, be used both for inputting and outputting sound.

In some embodiments, the one or more electroacoustic transducers 222, 224 may be regular microphone(s) and/or speaker(s). Thus, they may not necessarily have to be placed against the body tissue of the user.

In an embodiment, the electroacoustic transducer 222, 224 comprises a bone conductor transducer. Using a bone conductor transducer configured to output sound as an example, the bone conductor transducer may be configured to be placed against the body tissue of the user. Thus, it may vibrate sound to the body tissue of the user. The sound may travel in the body tissue of the user (e.g. skin, bones) towards inner-ear or internal ear of the user. Thus, the user may hear the outputted sound by the bone conductor transducer. For example, the sound may travel via cranial bones of the user. Bone conductor transducer may be beneficial in the swimming environment. That is, outputted sound may be more easily heard underwater as the sound does not necessarily have to travel via water-interface to reach user's ear(s). Therefore, outputted notifications, by the audio module 220, may be heard by the user in a variety of situations.

The sensor unit 210 and the audio module 220 may be configured to enable communication between the sensor unit 210 and the audio module 220. In some embodiments, as discussed above, the sensor unit 210 and the audio module 220 may form the device 200. However, in some embodiments, the sensor unit 210 and the audio module 220 may be separate entities. In some cases, the sensor unit 210 and the audio module may be connected to each other via a wire or via air-interface (e.g. wireless communication).

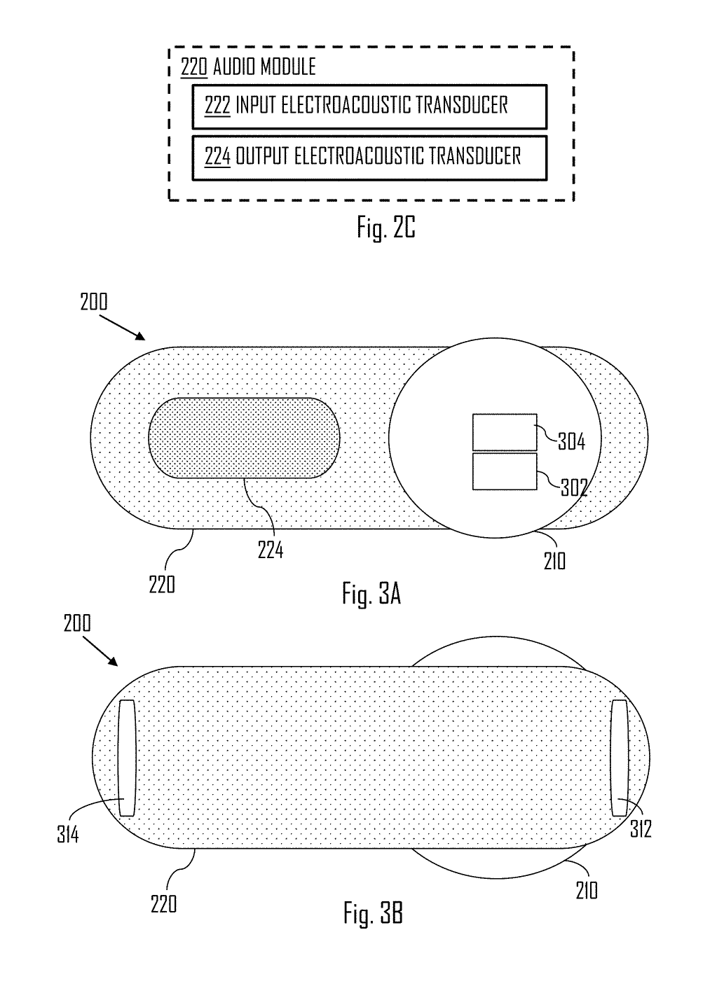

FIGS. 3A to 3C illustrate some embodiments. Referring to FIG. 3A, the device 200 may be shown. The device 200 may comprise the sensor unit 210. In some embodiments, the device 200 comprises the sensor unit 210 and the audio module 220. The sensor unit 210 may comprise an optical cardiac activity circuitry comprising one or more light emitting diodes (LEDs) 302 and one or more optical cells 304 configured to detect light (e.g. image sensors). The LED(s) 302 may emit light towards body tissue of the user and the one or more optical cells 304 may detect light emitted from the body tissue of the user (e.g. traveled through blood veins of the user). The optical cardiac activity circuitry may determine one or more values (e.g. heart rate, HRV, HBI) characterizing the cardiac activity of the user from the detected light. The one or more LEDs may be single color LEDs and/or RGB (Red Green Blue) LEDs, for example. The LED(s) 302 and the optical cell 304 may be placed against the body tissue of the user (e.g. skin).

In an embodiment, the optical cardiac activity circuitry comprises two LEDs 302 and one optical cell 302. The optical cell 304 may be situated between the two LEDs 302.

In an embodiment, the optical cardiac activity circuitry is comprised in the cardiac activity circuitry 214 or is the cardiac activity circuitry 214.

Referring to FIG. 3A, the audio module 220 may comprise the electroacoustic transducer 224 (e.g. the bone transducer). The electroacoustic transducer 224 may be placed against the body tissue of the user and configured to output sound towards the body tissue of the user.

The device 200 may be configured to be attached to the swimming goggles 108. The attachment may be configured to be detachable. Referring to FIG. 3B, the device 200 may be attached to the swimming goggles 108 using the one or more attachment elements 312, 314 comprised in the audio module 220. For example, the one or more attachment elements 312, 314 may comprise one or more openings (e.g. slots) for attaching the device 200 or the audio module 220 to the strap 182 of the swimming goggles 108. Thus, the strap 182 may be threaded in to the one or more openings 312, 314. In an embodiment, the slots 312, 314 comprise an opening for the strap and one more opening enabling the threading to the strap. In an embodiment, the audio module 220 comprises at least one attachment element 312, 314 for attaching the device 200 to the strap of the swimming goggles 108.

In an embodiment, the FIGS. 3A and 3B illustrate opposite sides of the device 200. Thus, the attachment elements 312, 314 may be configured on the opposite side compared to the cardiac activity circuitry 214 of the sensor unit 210. This may create more force towards the body tissue of the user, and thus enhance the cardiac activity measurement.

Referring to FIG. 3C, the sensor unit 210 may be detachably attachable to an audio module 220. Therefore, for example, as the audio module 220 may be attached to the swimming goggles 108 and the sensor unit 210 may be attached to the audio module 220, the sensor unit 210 may thus also be attached to the swimming goggles 108. In another embodiment, the sensor unit 210 is directly attachable to the swimming goggles 108. For example, the sensor unit 210 may comprise one or more slots for attaching the sensor unit 210 to the strap of the swimming goggles 108.

In an embodiment, the audio module 220 comprises a slot 310 for the sensor unit 210. The sensor unit 210 may be placed at least partially into the slot 310. The slot 310 may be adapted and dimensioned such that the sensor unit 210 is detachably attachable to the audio module 220.

It needs to be noted that although the attachment of the device 200 is described above as attachment to the strap of the swimming goggles 108, other attachment means may be employed. One example of this may be to attach the device 200 with adhesive to the swimming goggles 108. In another example, the swimming goggles 108 may comprise a holster for the device 200. The holster may be situated on the inner surface of the swimming goggles 108 (e.g. in the inner surface of the strap that is placed against the head of the user). In a yet another example, the swimming goggles 108 may comprise one or more frames, wherein the device 200 is configured to be attached to the one or more frames.

In an embodiment, the swimming goggles 108 comprise the audio module 220 and/or the sensor unit 210. Thus, the device 200 may be integrated directly to the swimming goggles 108.

In an embodiment, the sensor unit 210 comprises one or more antennas, as was also described above. In an embodiment, the sensor unit 210 comprises at least one coil 392 configured to transmit and/or receive electromagnetic radiation or energy. In an embodiment, the at least one coil 392 is situated on the edge area of the sensor unit 210, as shown in FIG. 3C. The at least one coil 392 may be used to transmit, for example, the physical activity data from the sensor unit 210 to one or more external devices and receive data as radiation from external devices.

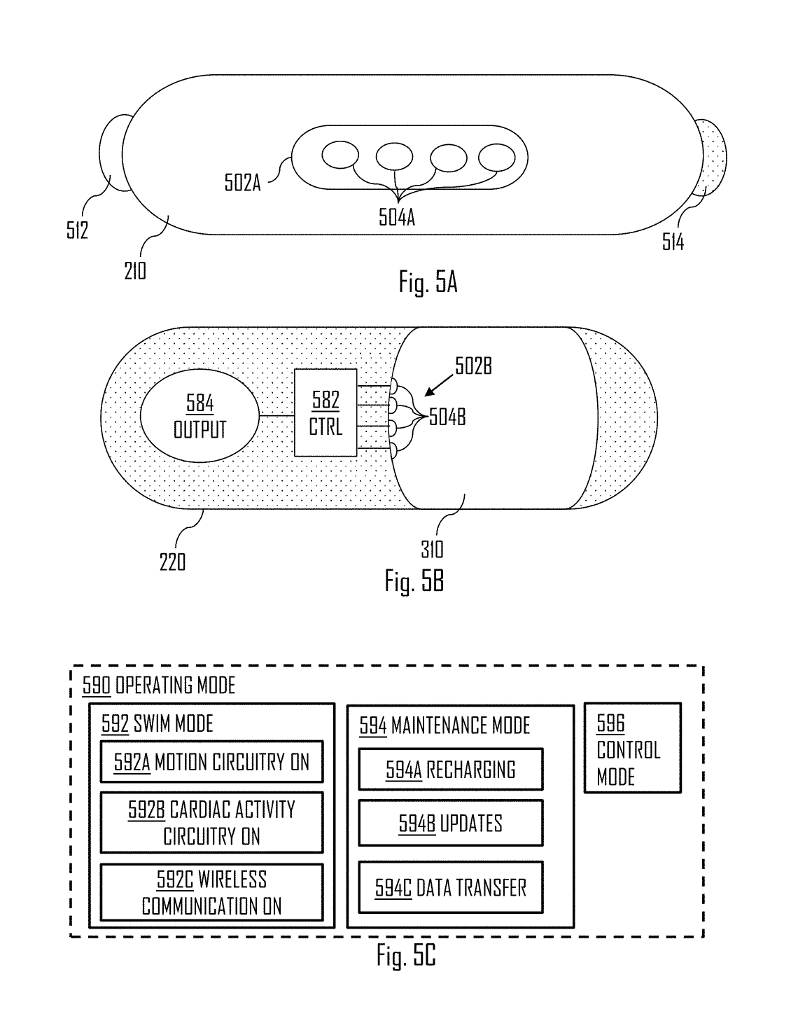

FIGS. 5A to 5B illustrate some embodiments. Referring to FIGS. 5A to 5B, the sensor unit 210 may comprise a first interface element 502A configured to enable electrical coupling of the sensor unit 210 to the audio module 220. The audio module 220 may comprise a second interface element 502B configured to enable electrical coupling of the audio module 220 to the sensor unit 210. In an embodiment, the sensor unit 210 comprises the first interface element 502A and the audio module 220 comprises the second interface element 502B, the first and second interface elements 502A, 502B configured to enable electrical coupling of the sensor unit 210 with the audio module 220.

In an embodiment, the device 200 comprises an interface element. The interface element may comprise the first and/or second interface elements 502A, 502B depending on whether the device 200 comprises only the sensor unit 210 or both the sensor unit 210 and the audio module 220. However, if the device 200 comprises only the first interface element 502A, the external device, to which the sensor unit 210 may be connected via the first interface element 502B, may comprise a corresponding interface element (e.g. the second interface element 502B). The interface elements may thus be electrically coupled to each other, and thus enable data communication (e.g. control message causing the audio module 220 to produce sound) and/or energy transfer (e.g. operational voltage from the sensor unit 210 to the audio module 220).

In an embodiment, the first and second interface elements 502A, 502B are configured to enable the sensor unit 210 to be communicatively coupled with the audio module 220. Thus, for example, the sensor unit 210 may control the audio module 220. This may enable, for example, the sensor unit 210 to cause the audio module 220 to output sound notifications according instructions or control signaling from the sensor unit 210.

In an embodiment, the sensor unit 210 comprises a power source. The power source may be, for example, a rechargeable battery. The power source may be detachable or removable, or it may be that the power source is fixed to the sensor unit 210. The power source may provide operational voltage to the sensor unit 210.

In an embodiment, the power source of the sensor unit 210, the first interface element 502A, and the second interface element 502B are configured to provide operational voltage to the audio module 220. Thus, the interface elements 502A, 502B may enable the power source of the sensor unit 210 to be used also as the power source of the audio module 220.

In an embodiment, the operational voltage to the audio module 220 is transferred between the first and second interface elements 502A, 502B as alternating current (AC) voltage. Using AC voltage may bring some benefits. As the device 200 may be used in water environment, corrosion in the interface elements 502A, 502B may cause problems. However, using AC voltage may decrease electrolysis. In an embodiment, the frequency of the AC voltage is around 10 MHz or over 10 MHz. In an embodiment, the sensor unit 210 comprises a microcontroller configured to provide the AC voltage from the power source via the interface to the audio module 220. The microcontroller may be comprised in the processing circuitry, for example.

In an embodiment, the audio module 220 comprises a rectifier configured to receive the AC voltage via the interface elements 502A, 502B and to provide the audio module with the operational voltage. Thus, the rectifier may change the AC voltage to direct current (DC) voltage, and thus the operational voltage of the audio module 220 may be DC voltage.

In an embodiment, referring to FIGS. 5A to 5B, the first interface element 502A comprises one or more connectors 504A. The second interface element 502B may comprise one or more counterparts 504B for the one or more connectors 504A. For example, the connector(s) 504A may be female connectors and the counterpart(s) 504B may be male connectors. In an embodiment, the first interface element 502A comprises three or four connectors 504A and the second interface element 502B comprises three or four counterparts 504B, wherein for each connector 504A there is one counterpart 504B.

In an embodiment, the first and second interface elements 502A, 502B are USB connectors. In an embodiment, the first and second interface elements 502A, 502B are micro-USB connectors. Thus, the first and second interface elements 502A, 502B may provide a USB or micro-USB interface between the sensor unit 210 and the audio module 220. For example, one of the interface elements may be a male connector and the other interface element is a female connector.

One aspect of using the first interface element 502A is that the sensor unit 210, or more precisely the power source of the sensor unit 210, may be charged via the first interface element 502. For example, the first interface element 502A may be coupled with an external device having a counterpart for the first interface element 502A. One simple example of this may be the USB or micro-USB case, wherein the sensor unit 210 may be charged via the USB or the micro-USB connector or port.

In the case of the USB or micro-USB, the supply voltage from the external device may be DC voltage. Therefore, there may be an additional switching rectifier (e.g. a separate part) used between the DC power supply and the first interface element 502A, if the first interface element 502A is configured to transfer power as AC voltage. If the voltage is transferred to or from the sensor unit as DC voltage, there may not be need to use the additional switching rectifier. In an embodiment, the switching rectifier is configured such that the switching frequency is tuned to be at a resonance of USB or micro-USB cable. When the cable is on resonance frequency, it may operate as an energy storage where energy may be transferred to the sensor unit 210. This may enable current to flow from the charging cable to the sensor unit 210 as AC, and thus reduce or remove risk of electrolytic corrosion. In an embodiment, the sensor unit 210 comprises said switching rectifier.

In an embodiment, the sensor unit 210 comprise a multiplexer configured to enable outputting of energy from the sensor unit 210 to the audio module 220 and to enable recharging of the power source of the sensor unit 210. The multiplexer may change mode of the sensor unit 210. Examples of such modes may be maintenance mode (e.g. recharging) and power source mode (e.g. providing power to the audio module 220).

FIG. 5C illustrates an embodiment. Referring to FIG. 5C, the sensor unit 210 may comprise one or more operating modes 590. The operating mode(s) 590 may be controlled by the sensor unit 210 (e.g. processing circuitry, microcontroller, multiplexer(s)). The operating mode(s) 590 may comprise a swim mode 592, a maintenance mode 594, and/or sleep mode.

In an embodiment, the sensor unit 210 is configured to operate in and/or to enter the swim mode 590 when the sensor unit 210 is electrically coupled with the audio module 220 via the first and second interface elements 502A, 502B.

In an embodiment, the sensor unit 210 is configured to operate in and/or to enter the sleep mode when the sensor unit 210 is electrically disconnected from the audio module 220.

In an embodiment, the sensor unit 210 is configured to automatically shut down when the sensor unit 210 is electrically disconnected from the audio module 220.

In an embodiment, the sensor unit 210 is configured to operate in a maintenance mode 594 when the sensor unit 210 is electrically coupled with an external device via the first interface element 502A. For example, if the sensor unit 210 is coupled via USB or micro-USB element to an external device or power source, the sensor unit 210 may enter the maintenance mode 594.

Let us now look closer on what the different modes may comprise with reference to FIG. 5C. When the sensor unit 210 operates in the swim mode 592, the sensor unit 210 may be configured to be on, the motion circuitry 212 may be configured to be on (block 592A), the cardiac activity circuitry 214 may be configured to be on (block 592B), the wireless communication circuitry 216 may be configured to be on (block 592C), and/or the audio module 220 may be configured to be on.

In an embodiment, in the swim mode 592, the wireless communication circuitry 216 is configured to operate on the 5 kHz band. In an embodiment, in the swim mode 592, the wireless communication circuitry 216 is configured to operate only on the 5 kHz band. For example, if both Bluetooth and 5 kHz band are utilized, this may save valuable energy resources.

In an embodiment, in the swim mode 592, the wireless communication circuitry 216 is configured to utilize both the 5 kHz band and Bluetooth. By doing this, the probability of data transfer may be enhanced.

In an embodiment, when the sensor unit 210 enters the swim mode 592, the audio module 220 may output one or more notifications. Thus, the user may know that the device 200 is operating in the desired mode, for example. Different modes may be associated with a different notifications.

In an embodiment, entering the swim mode 592 by the sensor unit 210 causes the sensor unit 210 to automatically switch on, the motion circuitry 212 to automatically switch on, the cardiac activity circuitry 214 to automatically switch on, the wireless communication circuitry 216 to automatically switch on, and/or cause the audio module 220 to automatically switch on.

When the sensor unit 210 operates in the maintenance mode 594, the sensor unit 210 may be configured to be recharged (block 594A), receive data via the first interface element 502A from an external device (e.g. configuration data or updates as in block 594B), and/or transmit data to an external device via the first interface element 502A (block 594C). For example, the first interface element 502A may be used with the external element (e.g. USB cable) to transfer data between the external device and the sensor unit 210. In an embodiment, the data transfer starts automatically when the sensor unit 210 is communicatively connected via the first interface element 502A to the external device. The data transfer may happen via the first interface element 502A and/or via the wireless communication circuitry 216.

When the sensor unit 210 operates in the sleep mode, the motion circuitry 212 may be configured to be off, the cardiac activity circuitry 214 may be configured to be off and/or the wireless communication circuitry 216 may be configured to be off.

In an embodiment, entering the swim mode 592 by the sensor unit 210 causes the sensor unit 210 to automatically switch off, the motion circuitry 212 to automatically switch off, the cardiac activity circuitry 214 to automatically switch off, and/or the wireless communication circuitry 216 to automatically switch off.

In an embodiment, the operating mode(s) 590 comprises a control mode 596. In an embodiment, the sensor unit 210 is configured to operate in the control mode 596 when the sensor unit 210 is electrically coupled with an audio module (e.g. the audio module 220), wherein in the control mode 592 the sensor unit 210 is further configured to control said audio module to output sound (e.g. sound notifications).

The electrical coupling between the sensor unit 210 and said audio module may be possible via the first interface element 502A and a corresponding interface element in said audio module. For example, the sensor unit 210 and the audio module 220 may be electrically coupled using the first and second interface elements 502A, 502B.

In an embodiment, the control mode 596 comprises the swim mode 592. Thus, the sensor unit 210 may perform operations of the swim mode 592 when operating or in the control mode 596.

In an embodiment, the sensor unit 210 is configured to operate in a maintenance mode 594 when the sensor unit 210 is electrically coupled with an external device, wherein in the maintenance mode 594 the sensor unit 210 is further configured to be recharged by said external device, to transmit physical activity data to said external device, and/or to receive configuration data from said external device. For example, the sensor unit 210 may be electrically coupled to a laptop via a cable and the first interface element 502A. The recharging and/or the data transfer may happen via said electrical coupling. Said electrical coupling may happen, for example, via the first interface element 502A.

The sensor unit 210 may be configured to determine that the electrical connection to said external device enables power and/or data transfer. For example, the sensor unit 210 may determine a power input to the first interface element 502A. The power input may cause the sensor unit 210 to enter to the maintenance mode.

In an embodiment, the sensor unit 210 comprises a controller configured to change the operating mode 590 of the sensor unit 210. The controller may comprise a multiplexer, for example. The controller may be electrically coupled with the first interface element 502A. The controller may be configured to determine a power input via the first interface element 502A, and as a response cause the sensor unit 210 to operate and/or enter the maintenance mode 594. The controller may be configured to determine that the power input via the first interface element 502A stops, and as a response cause the sensor unit 210 to operate and/or enter some other mode (e.g. sleep mode) or cause the sensor unit to shut down. The controller may be configured to determine that the sensor unit 210 is electrically coupled via the first interface element 502A to an audio module (e.g. the audio module 220) and as a response, cause the sensor unit 210 to operate and/or enter the control mode 596.

It needs to be noted that the phrase electrical coupling may refer to wireless coupling or galvanic coupling. For example, magnetic induction may be used between the first and second interface element 502A, 502B. However, using the galvanic electrical coupling, the power transfer, data transfer and controlling may be more reliable. Further, operation of controller of the sensor unit may be more reliable. For example, the electrical coupling may happen via USB or micro-USB interfaces. Thus, for example, the first interface element 502A may be a USB or a micro-USB interface element configured to be attached to a corresponding USB or micro-USB interface of an audio module (e.g. the audio module 220) or to a corresponding USB or micro-USB interface of another external device (e.g. power source, portable electronic device 106).

Let us now discuss a bit more about the sound notifications that the audio module 220 may output. The sensor unit 210 and the audio module 220 may be configured such that the sensor unit 210 may cause the audio module 220 to output sound notifications. The sound notifications may, for example, be related to the physical activity performed by the user. For example, the sound notifications may be outputted and/or caused to be outputted on the basis of the measured physical activity data. Examples of such notifications may comprise a lap time, heart rate, heart rate zone, calorie consumption, and/or distance traveled or swam.

In an embodiment, the audio module 220 is configured to output a notification indicating a heart rate of the user. Other cardiac activity characterizing values, such as HRV and/or HBI, may also be outputted. For example, current heart rate of the user may be indicated.

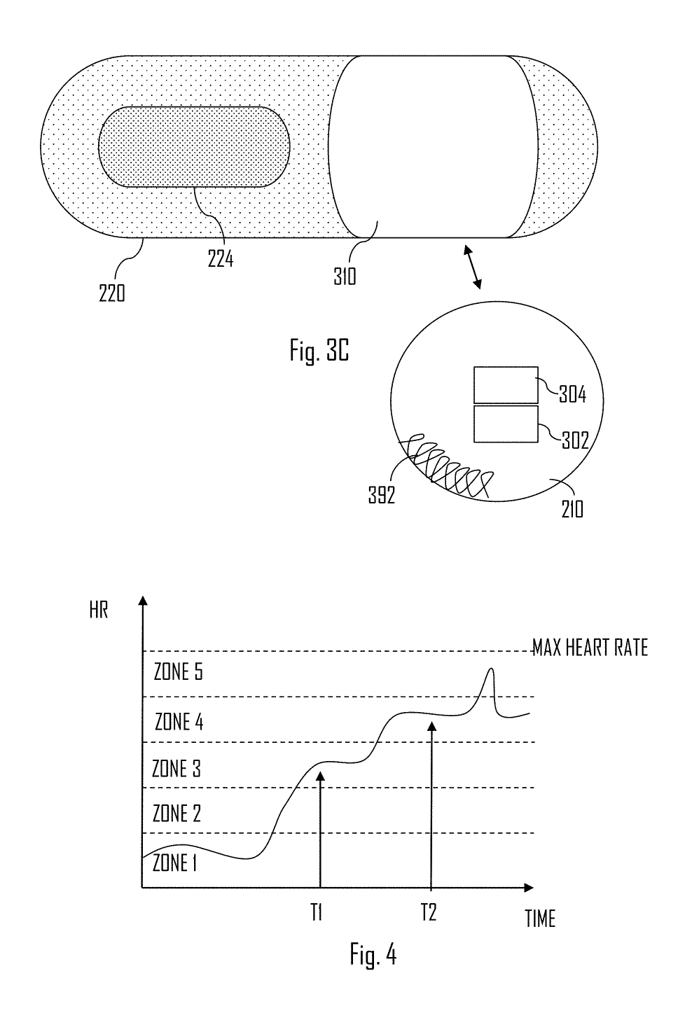

In an embodiment, the audio module 220 is configured to output a notification indicating a heart rate zone of the user. Example of heart rate zones may be shown in an embodiment of FIG. 4. There may be a plurality of heart rate zones. Referring to FIG. 4, five heart rate zones of the user may be shown (number of zones may vary, but five heart rate zones may be one beneficial embodiment). Heart rate of the user in a function of time is also shown. For example, at time T1 the audio module 220 could output a notification indicating heart rate zone 3. At time T2 the audio module 220 could output a notification indicating heart rate zone 4. Thus, for example, current heart rate zone could be indicated. In some embodiments, a target heart rate zone is indicated by the audio module 220. For example, if the user has selected a training session targeting highest fat burn, the audio module 220 may indicate the target heart rate zone if the user is not on that zone and/or if the user is on or enters that zone. It needs to be understood that the sensor unit 210 may be configured to cause the outputting of the notification on the basis of the measured heart rate, for example.

In an embodiment, the audio module 220 is configured to output a notification indicating a maximum heart rate of the user. User may thus know when he/she has reached his/her limit.

In an embodiment, the heart rate zones (e.g. indicated in FIG. 4) are user specific. The sensor unit 210 may calculate and/or acquire the user specific heart rate zones. The calculation may be based on user parameters, such as age, gender, results of one or more physical activity tests, height and/or weight. The user parameters as well as other configuration data may be received, by the sensor unit 210, from an external device, such as the wrist device 102, the portable electronic device 106, and/or the server 114.

In an embodiment, the heart rate zones are swimming style specific. In an embodiment, the heart rate zones are swimming style specific and user specific. Thus, the heart rate zones may vary between different swimming styles (e.g. breaststroke, backstroke, butterfly stroke) and/or users. The sensor unit 210 may be configured to determine (e.g. based on the motion data obtained using the motion circuitry and/or one or more external sensor devices or based on user input) swimming style of the user (e.g. the current swimming style) and configure the heart rate zones for the determined swimming style. Further, the configured heart rate zones may vary between users for the same swimming style.

As was discussed above, the notifications by the audio module 220 may be controlled by the sensor unit 210, for example. Thus, the sensor unit 210 may perform measurements using the motion circuitry 212 and/or the cardiac activity circuitry 214. Based on the measurements, the sensor unit 210 may determine when and what kind of notification should be outputted by the audio module 220. Therefore, in some embodiments, the sensor unit 210 comprises a processing circuitry configured to process the measurement data obtained using the motion circuitry 212 and/or the cardiac activity circuitry 214. The processing circuitry may comprise one or more processors and one or more memories, for example.

In an embodiment, the processing circuitry is configured to obtain the motion data and/or the cardiac activity data, as well as other physical activity data, and to process the obtained data into one or more metrics characterizing the physical activity performed by the user. Examples may comprise calorie consumption, distance traveled, intensity of training, speed, velocity, and the like. The processing circuitry may be configured to perform other actions of the sensor unit 210. Implementation of using the processing circuitry (e.g. at least one processor and memory with program code and/or Application Specific Integrated Circuit (ASIC) to give two examples) to perform different action of the sensor unit 210 is clear to a skilled person.

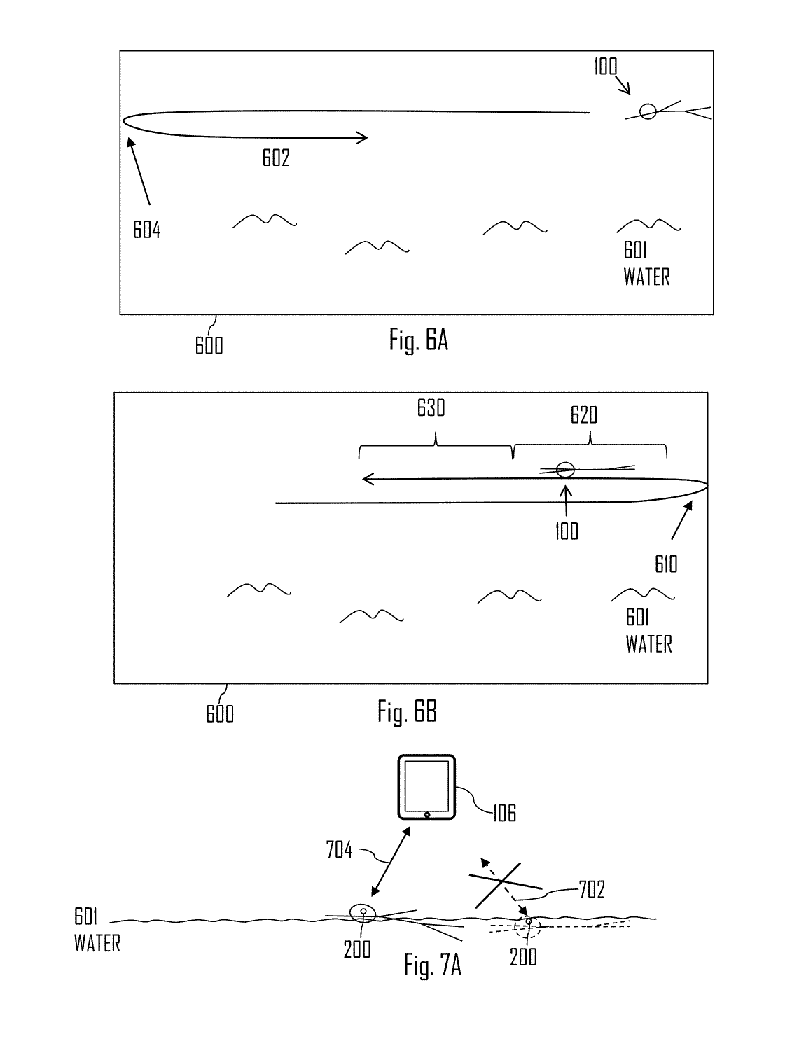

FIGS. 6A to 6B illustrate some embodiments. Referring to FIGS. 6A to 6B, a swimming pool 600 and water 601 may be shown. The user 100 may swim in the pool 600. This illustrates an example system or environment to which at least some embodiments may be applied to. However, it is evident that the embodiments are not necessarily restricted to this example system.

Referring to FIG. 6A, the device 200 and/or the sensor unit 210 may be configured to detect a turn 604 by the user during swimming and to cause the audio module 220 to output a notification indicating a recorded time. The turn 604 may be detected using the motion data obtained using the motion circuitry 212 and/or from motion data obtained using on or more external motion sensors. For example, the motion data characterizing the motion of the user may be used to determine overall motion 602 of the user. Thus, it may be determined when the user turns during swimming. Also, if GPS or GLONASS is employed, the turn may be detected from that data. This may be applicable also for indoors, if the satellite signal quality is high enough.

In some embodiments, the turn is determined and/or detected, by the sensor unit 210, using one or more gyroscopes, one or more accelerometers, and/or one or more magnetometers.

In an embodiment, the sensor unit 210 is configured to measure the motion of the user using the motion circuitry 212. The sensor unit 210 may transmit motion data to the wrist unit 102 using, for example, the 5 kHz band. The wrist unit 102 may then determine the turn based at least partly on the received motion data. The wrist unit 102 may then cause outputting of one or more notifications via the wrist unit 102 and/or some external device. For example, the wrist unit 102 may cause the audio module 220 to output notifications. Further, the wrist unit 102 may comprise one or more motion sensor configured to measure motion of the user. The wrist unit 102 may also be connected to the external sensor device(s) 104. Thus, the wrist unit 102 may employ motion data from multiple sensors to determine the turn(s) during swimming.

In an embodiment, the recorded time outputted by the audio module 220 comprises at least one of a lap time of the user, a pool length time of the user, a total swimming time. For example, the lap length may be configured (e.g. configuration data) by the user or may be preconfigured to the device 200 or to the wrist unit 102. In one example, the lap time may be two times the pool length.

In an embodiment, the recorded time is outputted as an absolute value (e.g. 1 minute 20 seconds). In an embodiment, the recorded time is outputted as a proportional value (e.g. ten seconds behind the target value). In an embodiment, the user may configure via web service, on the device 200, and/or on the wrist unit 102 in which form the recorded time is outputted. The configuration data may be transmitted to the sensor unit 210, and the sensor unit 210 may then change the output type accordingly. This configuration data may comprise other data also, such as pool length and/or lap length configuration.

In an embodiment, the device 200 or the wrist unit 102 is configured to determine when the user has stopped swimming and as a response, to cause outputting of a summary of the exercise via the audio module 220 or on the wrist unit 102 (e.g. display or speaker). The determination may be based on the motion data of the user. For example, the device 200 or the wrist unit 102 may determine that the user has stopped swimming based on detection that the user is on a substantially upright position (e.g. gyroscope data). The outputted summary may comprise, for example, indication of total time of the swimming exercise, calories consumed, and/or total distance swum.

According to an embodiment with reference to FIG. 6B, the device 200 is configured to determine a glide phase 620 of a turn and to cause the audio module 220 to output the notification indicating the recorded time during the glide phase 620. Looking at the FIG. 6B, the turn may comprise at last turning 610 and the glide phase 620. The swimming may then continue to stroke phase 630. These different swimming phases may be determined by the sensor unit 210. It may be beneficial to output the notification(s) during the glide phase 620, as the user may be less occupied with the swimming. In other words, the user may have more time to concentrate on the outputted notification(s). The glide phase 620 may be determined from the motion data obtained using the motion circuitry 212 and/or one or more external sensors.

In an embodiment, the detecting the turn, by the device 200, is based on at least one of the motion data obtained using the motion circuitry 212, data obtained using one or more external sensor devices worn by the user. In an embodiment, the detecting the turn is based on an external sensor device configured to be worn on the chest area of the user.

In an embodiment, the device 200 is configured to determine the turn during the swimming from a maximum acceleration value detected from the motion data. As the user turns, acceleration (e.g. negative and/or positive) may increase to a peak value. This may indicate the turn. This may be especially beneficial for a motion sensor that is worn on the torso of the user (e.g. chest area). This is due to the fact that acceleration of the torso may be more predictable compared to acceleration of limbs. Also head sensor (e.g. motion circuitry 212) may be a good measurement point. However, algorithm(s) in the sensor unit 210 may be configured to detect the turn also from the motion sensor(s) worn on the limbs of the user.

It needs to be noted that the motion data of the user may be obtained from a longer period than just for one turn. Thus, this history data may be used to determine similar patterns in the measured data. Therefore, the history data may be used to detect turn(s).

Also, in some embodiments, the device 200 is configured to determine at least two turns and to measure time between two consecutive turns. The device 200 may then cause outputting of said measured time after a turn or during the glide phase 620, for example.

In an embodiment, the device 200 is configured to determine when the user turns (e.g. arrow 610). The device 200 is further configured to wait for a predetermined time (e.g. 0.5 seconds, 1 second, 2 seconds) before causing the outputting of the recorded time. This time may be also referred to as a guard time.

In an embodiment, the sensor unit 210 comprises a teaching mode. In the teaching mode, the sensor unit 210 may be configured to determine a motion data pattern for a swimming turn(s). Thus, in the swim mode, the sensor unit 210 may use the determined motion data pattern for the swimming turn(s) to detect swimming turns.

It also needs to be noted that embodiments described in relation to FIGS. 6A and 6B, and also embodiments describing outputting notifications may be performed without the sensor unit 210. For example, the wrist unit 102 may comprise same or similar sensors as the sensor unit 210. The wrist unit 102 may cause the device 200 (i.e. audio module 220) to output notifications. The device 200 may thus comprise the wireless communication circuitry 216 or similar for receiving commands from the wrist unit 102. Also, it may be possible that the device 200 comprises the sensor unit 210, but the calculations may still be performed in the wrist unit 102. However, the wrist unit 102 may not be necessary for performing the above described embodiments.

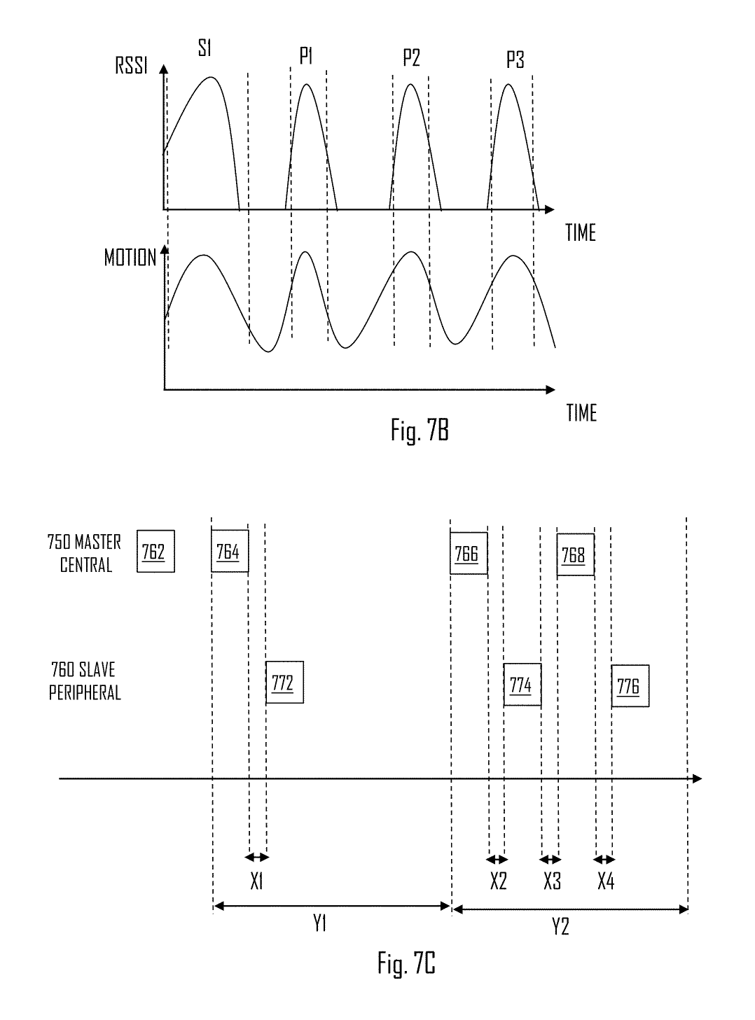

Let us then look closer on some embodiments shown in FIGS. 7A to 7C. A wearable device, such as the device 200, the wrist unit 102, and/or the portable electronic device 106, may be configured to transfer data with external apparatuses. Looking at the example of FIG. 7A, the device 200 may transmit data with the portable electronic device 106 and/or with the wrist unit 102. When the wearable device is at least partially underwater, the signal or data transmission quality may get weaker or the communication link between the devices is nonexistent. That is, ability of the wearable device to transmit data with an external apparatus may get weaker or totally stop. This may be especially the case for Bluetooth or WLAN transmissions, but may also affect the transmissions on the 5 kHz band.

Therefore, the wearable device may be configured to determine at least one time period when the wearable device is at least partially above water during swimming. The wearable device may further be configured to transfer data with the external apparatus during the at least one time period. The determination of the at least one time period may be based on, for example, the motion data of the user (e.g. acquired using the motion circuitry 212 or some other motion sensor(s)) and/or measurement(s) by a wireless communication circuitry used for the data transfer. Also in an embodiment, the wearable device comprises a water sensor configured to detect when the wearable device is at least partially in the water. The water sensor may be based on, for example, conductivity measurement.

Looking at the example of FIG. 7A, when the device 200 is underwater or the wireless communication circuitry 216 is underwater, the data transfer 702 with the portable electronic device 106 may not be possible or may be weaker in quality. Therefore, the device 200 may determine that no data should be transmitted. However, when the device 200 or the wireless communication circuitry 216 is above water, the data transfer 704 may be possible or may be better in quality. Therefore, the device 200 may determine that data should be transmitted. Using this approach, the device 200 may save valuable resources, such as battery, as there may not be need to retransmit data so often.

Referring to FIG. 7B, a few examples about the determination by the wearable device may be shown. That is, the wearable device may be configured to determine from motion data (e.g. accelerometer data) the time periods when the wearable device is underwater and/or when it is above water level. For example, if the user is swimming backstroke, the hand in which the wrist unit 102 is worn may show a movement pattern from which the determination may be made. Also, if the device 200 is worn, head of the user may make a pattern from which the determination may be made. The wearable device may be configured to transmit data during the time periods when the wearable device is above water level. Thus, in an embodiment, the wearable device may be configured to determine that the device is above water level or not underwater, and as a response transmit data to an external device.

Further, as described, the measurement(s) by wireless communication circuitry may also be utilized. In an embodiment, the wearable device is configured to combine the motion data and the measurement data by the wireless communication circuitry. The wearable device may then determine, based on the combined data, the at least one time period when the wearable device is above water level. Referring to FIG. 7B as an example, the Received Signal Strength Indicator (RSSI) may correspond to motion data. Therefore, associating the measured RSSI value with motion data may enable the device 200 to use the motion data to estimate the time periods P1, P2, and/or P3 without additional RSSI measurements. So basically, the device 200 may try to transmit the physical activity data during the time periods P1, P2, and/or P3 based on the motion data obtained using the motion circuitry 212 and/or some other motion sensor(s).

To be even clearer, according to one example, the device 200 may first measure RSSI or similar value during period 51 or some other time period. This measurement may be used to determine the time during the period 51 when the RSSI is over a desired value. The device 200 may then determine corresponding period from the motion data to determine which kind of motion pattern corresponds to the RSSI values being over the desired value. Thus, the device 200 may determine the periods P1, P2, and/or P3 from the motion data during which the data should be transmitted (i.e. without additional RSSI measurements). The device 200 may transmit data during the periods P1, P2, and/or P3.

Although the examples with reference to FIG. 7B disclose RSSI measurement, the device 200 or the wireless communication circuitry 216 may be configured to perform additionally or instead some other signal strength measurement. Further examples of these measurements comprise Signal to Noise (SNR) ratio and path loss measurement. Similar association with the motion data may be made with said signal strength measurement.

In an embodiment, the device 200 or the wireless communication circuitry 216 is configured to perform signal strength measurement. Based at least partly on said measurement, the device 200 may be further configured to determine the at least one time period for transmitting the physical activity data and/or receiving data from an external device.

In an embodiment, the wearable device, such as the device 200, comprises a conductivity sensor. The conductivity sensor may be comprised in the sensor unit 210, for example. The conductivity sensor may be configured to measure conductivity of the environment. Thus, wearable device may determine when the wearable device is underwater and/or above water level based on the measurement by the conductivity sensor. In an embodiment, the wearable device comprises a water contact sensor. The water contact sensor may be used to determine when the wearable device is underwater and/or above water level. Thus, the wearable device may use these sensors to determine the appropriate time period(s) for data transfer.

In an embodiment, the wearable device is configured to cause the wireless communication circuitry of the wearable device (e.g. the wireless communication 216) to change transmission interval. This may happen, for example, when the said device determines that it is used during swimming or is in a water environment. In an embodiment, the transmission interval is changed when said device is in the swim mode 592. The changing of the transmission interval may comprise decreasing the transmission interval. That is, during swimming or in the swim mode 592, said device may transmit data with a shorter transmission interval. For example, the transmission interval may normally be one second, and when the wearable device is used during swimming, the transmission interval may be changed to 0.1, 0.2, 0.3, 0.4, or 0.5 seconds to name a few examples. In an embodiment, the transmission interval is bisected during swimming or when operating in the swim mode 592.

The transmission of data by the wearable device (e.g. the device 200) may be broadcasting or unicasting for example. In an embodiment, the transmission is performed using Bluetooth (e.g. BLE) communication. Thus, the unicasting may refer to link layer connection between the wearable device and an external device.

The external device described above may be the portable electronic device 106, for example. In one example, the portable electronic device 106 is used by a trainer of the user wearing the wearable device during swimming. Thus, the data communication between the wearable device and the portable electronic device 106 may be used to monitor performance of the user and also to transmit training instructions and/or configuration data to the wearable device by the portable electronic device 106. For example, the trainer may want to indicate something to the user. For example, the portable electronic device 106 may transmit a control message to the wearable device, wherein the wearable device may cause an outputting of an indication to the user. The indication may comprise, for example, indicating end of the training session. For example, if the wearable device is the device 200, the audio module 220 may be caused to indicate the received instructions, from the portable electronic device 106, to the user.

Further, as explained, the Bluetooth broadcast and/or link layer connection interval may be increased during swimming. Also, if the link layer connection is used, the wearable device may be configured to retransmit data block or packet to the external device (e.g. the portable electronic device) a plurality of times (e.g. 2 times, 3 times, 4 times). The retransmitting may be stopped if the external device indicates to the wearable device that the data block or packet is received successfully.

FIG. 7C shows an example of Bluetooth data transmission according to an embodiment. The wearable device describe above, such as the device 200 (i.e. the device 200 or the wireless communication circuitry 216 in the sensor unit or in the device 200), may be configured to broadcast and/or unicast data (e.g. link layer connection). Referring to FIG. 7C showing an example of using the link layer connection, the wearable device may be configured to act as a slave peripheral device 760 and the external device (e.g. the portable electronic device 106 or some other external device to which the physical data is transmitted to) may be configured to act as a master central device 750. In the broadcasting case (not shown in FIG. 7C), the slave 760 may change broadcasting event interval so that the master 750 may most probably receive the broadcasted data. Thus, in an embodiment, the wearable device is configured to control the broadcasting interval (e.g. decrease the broadcasting interval). The broadcasting may comprise transmitting, by the wearable device, the physical activity data in one or more advertising packets.

In the link connection case (shown in FIG. 7C), the master 750 may change connection interval and/or the slave 760 may change slave latency (e.g. retransmit data block or packet a plurality of times). Thus, the connection interval may be decreased and/or the slave latency may be increased.

Referring to FIG. 7C, the connection intervals may be indicated with Y1 and Y2, wherein Y1 and Y2 are 7.5 millisecond to 4 second long X1-X4 may refer to timing values which may be used to configure the timing of the link layer connection transmission.

In block 762, the master 750 (e.g. the portable electronic device 106) may transmit a connection request to the slave 760. In block 764, the master 750 may transmit a data request to the slave 760. The slave 760 may answer by transmitting a data block (block 772) if the data request is received. The data block may comprise physical activity data or may indicate that there is now data to be transmitted.

However, as explained, the connection interval and/or the latency of the link layer connection may be changed. Thus, in blocks 766, 768 data requests are transmitted with a shorter interval, and correspondingly data blocks 774, 776 may also be transmitted with a shorter interval. Thus, the master 750 may more reliably receive the data that it requests. For example, during Y1 if the slave 760 is underwater during block 764 and/or block 772, the master 750 may possibly not receive the requested data. However, during Y2 it may suffice that the slave 760 is above water during at least one of the blocks 766, 768 and during at least one of the blocks 774, 776. If the slave is above water only during block 768, the data transfer may require that the slave 760 is also above water during block 776. For example, the data packets 774, 776 may be the same data packet (e.g. master data packet 774, and backup data packet 776).

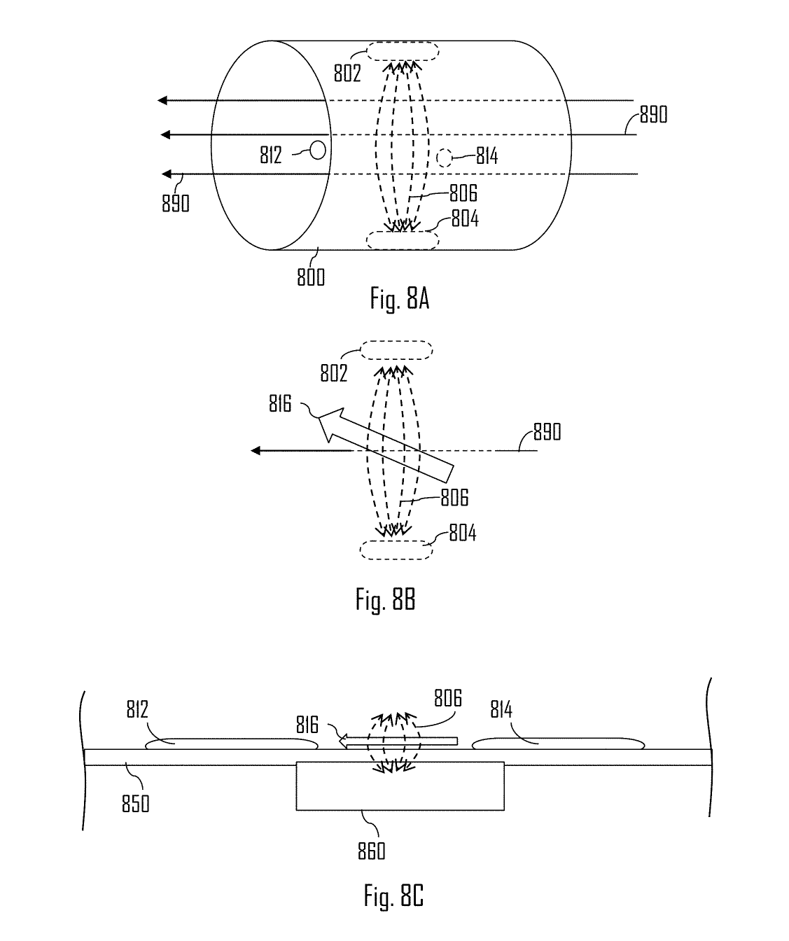

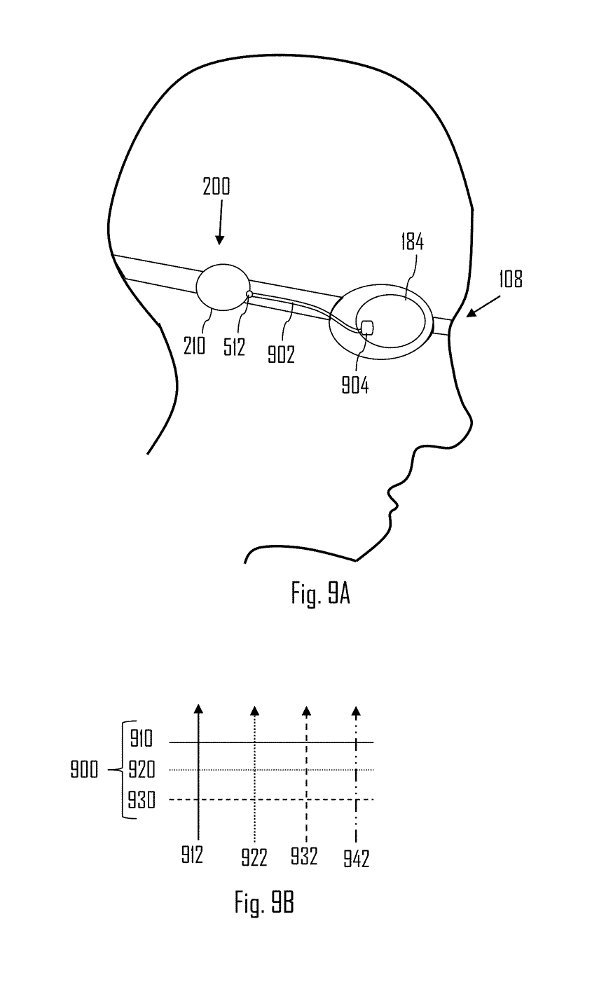

FIGS. 8A to 8B illustrate some embodiments. Referring to FIG. 8A, an electromagnetic flowmeter 800 may be shown. The wearable device, such as the sensor unit 210 or the device 20, may comprise the electromagnetic flowmeter 800. For example, the motion circuitry 212 may comprise the electromagnetic flowmeter 800. In an embodiment, the electromagnetic flowmeter 800 is comprised in an external sensor device configured to be worn by the user. For example, the electromagnetic flowmeter 800 and/or the external sensor device may be configured to be worn on the chest of the user. In an embodiment, said external sensor device is configured to measure cardiac activity of the user and swimming speed of the user.

The electromagnetic flowmeter 800 may be configured to measure swimming speed of the user. In an embodiment, the physical activity data transmitted from the device 200 to an external device comprises the swimming speed. In an embodiment, the device 200 or the sensor unit 210 is configured to cause the audio module 220 to output a notification indicating the swimming speed.

According to an embodiment and referring to FIG. 8A, the electromagnetic flowmeter 800 may comprise at least two electrodes 812, 814. The electromagnetic flowmeter 800 may be open from at least one area (e.g. two areas as shown in FIG. 8A). Thus, water 890 may flow through or in the electromagnetic flowmeter 800 when the user is swimming. The electromagnetic flowmeter 800 may further comprise at least one magnet 802, 804 causing a magnetic field 806 in the electromagnetic flowmeter 800. The water (i.e. conductive fluid) may flow through the magnetic field 806, and thus cause an electric field (not shown in FIG. 8A) perpendicular to the magnetic field 806 and the flow of the water 890. The electric field may cause an electric signal into the electrodes 812, 814. The electric signal may be measured, and thus the swimming speed of the user may be determined.

The induced voltage by the electric field may be calculated as follows: u=k*B*v*d, wherein u is the induced voltage (causing the electric signal to the electrodes 812, 814), B is the magnetic field strength, d is distance between the electrodes 812, 814, v is the velocity of the liquid flowing through the electromagnetic flowmeter 800, and k is a calibration factor. Thus, from this equation, the velocity v or speed may be calculated.

Referring to FIG. 8B, an electric field 816 may be shown together with the magnetic field 806 and the flow of the water 890. As shown in FIG. 8B, the electric field 816 (causing the induced voltage and electric signal), the magnetic field 806 (caused by at least one magnet, e.g. two magnets 802, 804) and the water flow 890 may be perpendicular to each other.

As an example, if there is a neodymium magnet causing a 1 tesla magnetic field in the electromagnetic flowmeter 800 and two electrodes 812, 814 are separated with 35 millimeters, 1 meter per second swimming speed may cause 35 millivolt signal into the electrodes 812, 814. The electromagnetic flowmeter 800 may further be calibrated (e.g. using the factor k) so that the correct swimming speed may be obtained. For example, GPS and/or GLONASS may be used to calibrate the electromagnetic flowmeter 800.

FIG. 8C illustrates the electromagnetic flowmeter 800 according to an embodiment. Referring to FIG. 8C, the electromagnetic flowmeter 800 may be an external sensor device or be comprised in the external sensor device or be comprised in the device 200. In an embodiment, said external sensor device is a cardiac activity transmitter configured to measure cardiac activity (e.g. heart rate) of the user and to transmit measured cardiac activity data to an external device (e.g. wrist unit 102).

Still referring to FIG. 8C, the electromagnetic flowmeter 800 may be obtained by utilizing a sensor device shown in FIG. 8C. Said sensor device may be, for example, a cardiac activity transmitter (e.g. heart rate belt) and/or the electromagnetic flowmeter 800. Said sensor device may comprise the at least two electrodes 812, 814, a substrate 850 (e.g. belt configured to enable said sensor device to be worn by the user), and an electronics module 860 (e.g. comprising a wireless communication circuitry and/or battery). Further, said sensor device may comprise at least one magnet. For example, the at least one magnet may be situated in or on the electronics module 860. For example, the at least one magnet may be situated in or on the substrate 850. The at least one magnet may be arranged and dimensioned such that it generates the magnetic field 816 that is at least partially between the at least two electrodes 812, 814. Thus, when said sensor device moves in water, water flow in the magnetic field 806 causes the electric field 816 between the at least two electrodes 812, 814. Thus, for example, the electronics module 860 may transmit speed data to an external device or at least determine speed of the user from the electric signal caused by the electric field 816. As explained above, the water flow, magnetic field 806, and the electric field 816 may be perpendicular to each other. However, according to some embodiments, the tubular structure, as shown in FIG. 8A, may not be necessary. Thus, as explained with reference to FIG. 8C, it may suffice that the water flows past the magnet(s) and/or the magnetic field 806 between the electrodes 812, 814.

Referring to FIG. 5A, the sensor unit 210 or the device 200 may comprise a user interface 514. The user interface 514 may comprise at least one button. In an embodiment, the user interface 514 comprises a display. The user interface 514 may be used to control the sensor unit 210 or the device 200. For example, the sensor unit 210 may switched on/off using the user interface 514.

Referring yet again to FIG. 5A, the device 200 and/or the sensor unit 210 may comprise one or more LEDs 512 configured to output visual notifications to the user. The one or more LEDs 512 may comprise different color LEDs or may be of the same color (e.g. emit light on same or different frequencies). In an embodiment, the one or more LEDs 512 are RGB LEDs. The one or more LEDs 512 may be situated in the sensor unit 210 or at least partially outside of the sensor unit 210.

Now referring to an embodiment of FIG. 9A, the one or more LEDs 512 may be shown. The device 200 and/or the swimming goggles 108 may further comprise one or more light guides 902, 904 configured to convey light emitted by the one or more LEDs 512 to one or more lens areas 184 of the swimming goggles 108. Thus, the one or more light guides 902, 904 may be situated such that they are connected (i.e. physically connected) with the one or more LEDs 512. Further, the one or more light guides 902, 904 may be situated such that they are connected (i.e. physically connected) with the at least one lens area 184 or lens.

In an embodiment, the one or more light guides 902, 904 comprise one light guide 902, 904 configured to convey light from one or more LEDs 512 to a lens of the swimming goggles 108.

In an embodiment, the one or more light guides 902, 904 comprise at least two light guides 902, 904 configured to convey light from two or more LEDs 512 to both lenses of the swimming goggles 108.

In an embodiment, the one or more light guides 902, 904 comprise optical fiber 902 configured to convey the light emitted by the LED(s) 512. In an embodiment, the optical fiber 902 is an optical fiber cable 902.

In an embodiment, the one or more light guides 902, 904 comprise an optical element 904 configured to be attached to the one or more lens areas 184 and to emit the light conveyed by the one or more light guides 902, 904 to the one or more lens areas 184. The attaching may be performed using an adhesive, for example.

In an embodiment, the one or more light guides 902, 904 are an integral part of the swimming goggles 108.

In an embodiment, the optical element 904 is situated on the inner surface of the at least one lens area 184.

In an embodiment, the optical element 904 is situated on the outer surface of the at least one lens area 184.

In an embodiment, the optical element 904 is situated in the at least one lens area 184. That is, the optical element 904 may be situated inside the lens structure (e.g. between two films of the lens).

In an embodiment, the one or more light guides 902, 904 penetrate through the swimming goggles 108 and/or the at least one lens area 184. This may enable the one or more light guides 902, 904 to be brought from the outer surface of the swimming goggles 108 on the inner surface of the at least one lens area 184 or lens. The penetration area may further comprise sealing to prevent water flowing into the goggles 108. For example, glue may be used for the sealing.

The visual notifications outputted by the one or more LEDs 512 and possibly conveyed by the one or more light guides 902, 904, may indicate similar things as the sound notifications outputted by the audio module 220. Thus, for example, heart rate, heart rate zone, speed, velocity, and/or direction may be indicated. In an embodiment, the visual notifications indicate a physiological parameter of the user. In an embodiment, the visual notifications indicate at least one value (e.g. heart rate, calorie consumption, heart rate zone and the like) characterizing the physical activity performed by the user. In an embodiment, the visual notifications relate to the physical activity performed by the user. In an embodiment, the visual notifications indicate physical activity data obtained using the sensor unit 210 or using some external sensor devices.

FIG. 9B illustrates an embodiment. Referring to FIG. 9B, the one or more light guides 902, 904 may comprise a filter element 900 configured to filter the light conveyed by the one or more light guides 902, 904. In an embodiment, the filter element 900 is situated in a head of the at least one light guide 902, 904. Said head may be situated at the end area of the one or more light guides 902, 904 which attaches to the at least one lens area 184. Thus, for example, the filter element 900 may be situated in the optical element 904 or between the optical element 904 and the optical fiber 902.

In an embodiment, the filter element 900 comprises one or more sub-filters 910, 920, 930 configured to filter light. For example, each filter sub-filter 910, 920, 930 may filter light that has a specific frequency or frequency area. For example, the first sub-filter 910 may filter substantially red light 912. For example, the second sub-filter 920 may filter substantially green light 922. For example, the third sub-filter 930 may filter substantially blue light 932. This may mean that the first sub-filter 910 may be configured to enable other frequencies of light to pass through it, but filters the substantially red light.

Using the filter element 900, it may be possible to display visual patterns on the swimming goggles. More particularly, it may be possible to display certain visual patterns on the at least one lens area 184.

In an embodiment, each sub-filter 910, 920, 930 filters light on a certain frequency area. For example, the first sub-filter 910 may be configured to filter light on a first frequency area. For example, the second sub-filter 920 may be configured to filter light on a second frequency area, wherein the first and second frequency areas are not at least partially overlapping.

In an embodiment, the first sub-filter 910 does not substantially filter light on the second frequency area. In an embodiment, the second sub-filter 920 does not substantially filter light on the first frequency area.

For example, the first sub-filter 910 may be configured to filter red light (not other frequencies) such that red light forms a first visual pattern on the at least one lens area 184. On the other hand, the second sub-filter 920 may be configured to filter green light (not other frequencies) such that green light forms a second visual pattern on the at least one lens area 184. Thus, for example, using three different sub-filters, different patterns may be displayed using different colors.

In an embodiment, the filter element 900 comprises more than three sub-filters. For example, there may be one sub-filter for yellow light. Also, it may be possible that the sub-filters are each configured each to filter white light 942, such that the white light 942 forms a specific pattern on the at least one lens area 184.

In an embodiment, the plurality of sub-filters 910, 920, 930 are situated on top of each other. This may make the displayed visual patterns a bit darker as in reality each sub-filter may filter all visible frequencies of light. However, the pattern(s) may still be formed as discussed above.

In an embodiment, the plurality of sub-filters 910, 920, 930 are situated adjacent to each other.

In an embodiment, the filter element 900 is configured to filter light such that the at least one visual pattern is displayed as presence of light. In an embodiment, the filter element 900 is configured to filter light such that the at least one visual pattern is displayed as absence of light. These examples may mean that the visual pattern (e.g. an arrow) may be shown, for example, as a red arrow or as a black (or some other colored) arrow on a red surface.

The visual patterns described above may comprise, but not limited to, arrow(s) (e.g. direction arrows), text, numbers, motivational text (e.g. “go faster”, “good job”), and heart rate zone. For example, heart rate zone could be indicated with different colors (e.g. without using the filter element 900) or using the filter element 900 (e.g. red light would display zone 1, green light would display zone 2 and so on).

In an embodiment, the one or more LEDs 512 and the one or more light guides 902, 904 are configured to output heart rate zone of the user. In an embodiment, the one or more LEDs 512, the one or more light guides 902, 904, and the filter element 900 are configured to output heart rate zone of the user. For example, the sensor unit 210 may determine heart rate of the user based on the cardiac activity data of the user. Further, the heart rate zone may be determined. Thus, the sensor unit 210 may cause the one or more LEDs 512, the one or more light guides 902, 904, and/or the filter element 900 to output the heart rate zone of the user.

In an embodiment, the one or more LEDs 512 comprise the filter element 900 or at least part of the filter element 900 (e.g. one sub-filter element).

In an embodiment, the filter element 900 comprises at least one polarization element. The polarization element(s) may also be used to provide different visual patterns on the at least one lens area 184. The polarization element may comprise a polarization film. In an embodiment, the polarization element is configured such that direction of the polarization can be changed. For example, the sensor unit 210 may be configured to change the polarization direction (e.g. vertical, horizontal) using electrical configuration.

In an embodiment, the filter element 900 comprises at least one diffraction grating element. The diffraction grating element(s) may also be used to provide different visual patterns on the at least one lens area 184.

In an embodiment, the filter element 900 comprises at least one filter film. For example, the sub-filters 910, 920, 930 may comprise one or more such films. For example, the film may be colored. For example, the film may be made of plastic.

In an embodiment, the filter element 900 comprises one or more plastic films, wherein each of the one or more plastic films is configured to filter light on a certain wavelength. The plastic films may, for example, be colored.

In an embodiment, the filter element 900 is referred to as a light filtering element 900. The light filtering element 900 may filter visible light on at least one frequency area.

It needs to be noted that although it is described that the filter element 900 filters light having a certain frequency, this may mean the same thing as filtering light having a certain wavelength. For example, red light may be have wavelength between 625-740 nanometers (i.e. 480-405 terahertz). For example, green light may have wavelength between 520-565 nanometers (i.e. 580-530 terahertz). Thus, for example, the first sub-filter 910 may filter light having a wavelength between X-Y, and the second sub-filter 912 may filter a wavelength between Z-A, to name a few examples.

In an embodiment, the one or more light guides 902, 904 are adapted and dimensioned to convey light emitted by the one or more LEDs 512 to a field of vision of the user. For example, the light may be conveyed to the lens or lens area of the swimming goggles 108.

In an embodiment, the one or more light guides 902, 904 comprise a lens element configured to change color of the lens according to the light emitted by the one or more LEDs 512. For example, an optical coating may be used in the lens(es) of the swimming goggles 108. Thus, the light may be dispersed substantially to the whole lens area.

In an embodiment, the one or more light guides 902, 904 comprise an optical fiber cable. The optical fiber cable may be coated with substantially light impermeable material, such as dark plastic or polymer in general. The optical fiber cable may be made of plastic, for example.

In an embodiment, the optical fiber cable is adapted and dimensioned to be attached to the at least one lens area of the swimming goggles 108. Thus, there may not be a need for an additional optical element in order to convey the light from the one or more LEDs 512 to the lens area(s).

In an embodiment, the optical fiber cable is welded or bonded to the swimming goggles 108. For example, ultraviolet (UV) bonding may be used.

In an embodiment, the optical fiber cable is adapted and dimensioned to be positioned in a groove or a hole in the swimming goggles 108. Thus, the optical fiber may be attached to the swimming goggles by mechanical force. Adhesive, such as glue, may be used to make the attachment more robust.

Let us then discuss a bit about the different notifications which may be outputted by the audio module 220 and/or the one or more LEDs (with the one or more light guides 902, 904 and/or the filter element 900). In an embodiment, the audio module 220 is configured to output sound notifications. The sound notifications may comprise music, speech, and/or alarms, to name a few examples.

In an embodiment, the audio module 220 comprises a speech synthesizer (also referred to as a speech computer). The speech synthesizer may be configured to generate audible sound (e.g. speech) from text. Thus, for example, the audio module 220 may be configured to generate synthetized speech. Thus, for example, heart rate or heart rate zone may be indicated with speech. Also, the audio module 220 may be configured to output motivational messages, such as “increase tempo” or “good job, keep up the good work”. The synthesized text may be received from the sensor unit 210 or may be generated by the audio module 220, for example.

In an embodiment, the sensor unit 210 may determine a heart rate zone of the user based on the cardiac activity data. Based on the determination, the sensor unit 210 may cause the audio module to output an indication. The indication may comprise one of indication to increase swimming tempo, indication to maintain current swimming tempo or indication to decrease swimming tempo. Said indication may be indicated as synthetized speech, for example.

In an embodiment, the speech synthesizer is configured to produce sound from one or more preconfigured text files. These text files may be comprised in the audio module 220 and/or received from the sensor unit 210, for example.

In an embodiment, the audio module 220 and/or the sensor unit 210 comprises one or more audio files. The audio files may be preconfigured to the audio module 220 and/or to the sensor unit 210, for example. The audio module 220 may output sound according to the audio files. For example, the sensor unit 210 may cause said outputting. For example, the sensor unit 210 may select the audio file to be outputted among a plurality of audio files, and cause the audio module to output sound according to the selected audio file. The audio files may be related to the physical exercise performed by the user. For example, one audio file may indicate “increase tempo”, whereas another may indicate “decrease tempo”.

In an embodiment, the audio module 220 comprises a haptic element configured to indicate notifications as vibration. Thus, vibration, sound, and/or visual notifications may be outputted by the device 200.

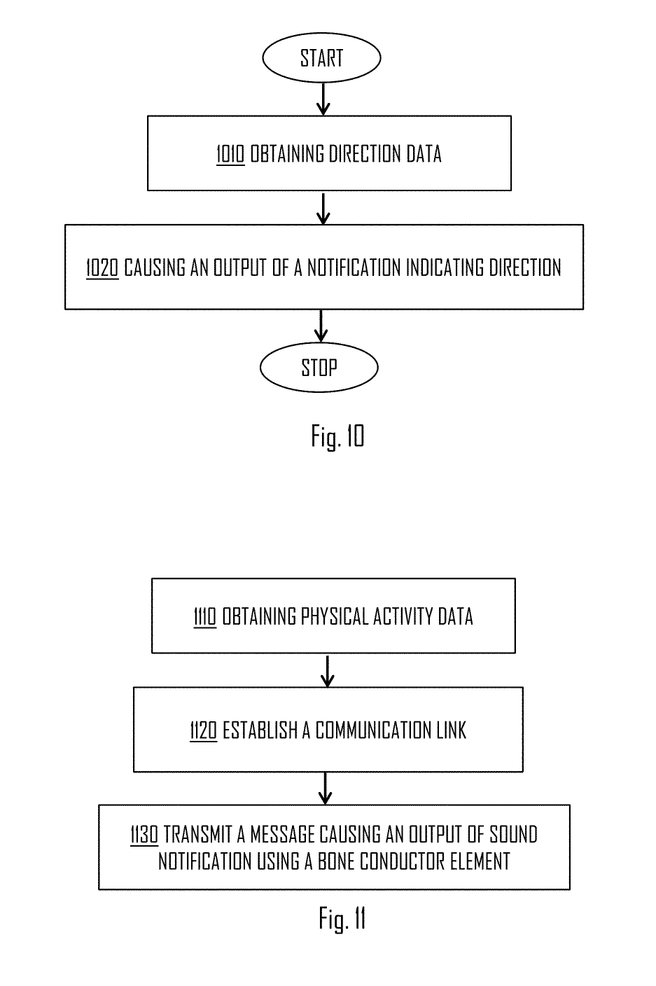

FIG. 10 illustrates yet another embodiment. Referring to FIG. 10, a wearable device, such as the device 200, the sensor unit 210, and/or the wrist unit 102, may obtain direction data (block 1010). The direction data may be obtained using magnetometer(s) and/or satellite positioning circuitry. For example, the sensor unit may use the motion circuitry 212 to obtain the direction data. The direction data may indicate a direction from a current location or a starting location to a target location. For example, if two locations are known, direction between the two may be determined. The direction may thus be determined between two buoys, for example.

In block 1020, the wearable device may cause an output of a notification indicating the direction data or the direction. For example, the audio module 220 may be caused, by the sensor unit 210 or the wrist unit 102, to indicate the direction. For example, the at least one LED 512 may be caused, by the sensor unit 210 or the wrist unit 102, to indicate the direction. For example, a display (e.g. comprised in the wrist unit 102, in the swimming goggles 108 or in some other device) may be caused, by the sensor unit 210 or the wrist unit 102, to indicate the direction.

Let us discuss a bit further how the direction may be indicated according to some embodiments. In an embodiment, the sensor unit 210 is configured to cause the one or more LEDs 512 to emit light according to the direction data or the direction. The one or more LEDs 512 may indicate whether the user deviates from the direction to right, to left, or is going to the right direction. For example, one color or a pattern (e.g. by the filter element 900) may indicate that the user deviates from the direction to left. For example, another color or another pattern (e.g. by the filter element 900) may indicate that the user deviates from the direction to right. Further, another color or pattern or absence of pattern may indicate that the user is substantially on the right course or going to right direction. For example, deviation to left may be indicated with color red, deviation to right may be indicated with color yellow, and right direction may be indicated with color green.

In an embodiment, referring to FIG. 11, there is provided a method for outputting notifications to a user during swimming, the method comprising: obtaining, by a device, at least one of a motion data of the user using a motion circuitry, cardiac activity data of the user using a cardiac activity circuitry (block 1110); establishing a communication link with an audio module comprising a bone conductor element (block 1120); and transmitting a message to the audio module causing the bone conductor element to output a sound notification concerning at least one of the motion data of the user, the cardiac activity data of the user (block 1130). The device may be the sensor unit 210 or the wrist device 102, for example. The audio module may be the audio module 220, for example.

According to yet another embodiment, the apparatus (e.g. the device 200, the sensor unit 210 and/or the audio module 220) carrying out the embodiments comprises a circuitry including at least one processor and at least one memory including computer program code. When activated, the circuitry causes the apparatus to perform at least some of the functionalities according to any one of the embodiments, or operations thereof.

In an embodiment, an apparatus carrying out the embodiments comprises means for performing the method according to any one of the embodiments, or operations thereof.

In an embodiment, there is provided a computer program product comprising program instructions which, when loaded into an apparatus, execute the method according to any one of the embodiments, or operations thereof. In an embodiment, there is provided a computer readable medium comprising said computer program.

In an embodiment, the device 200 comprises a circuitry including at least one processor and at least one memory including computer program code, wherein the circuitry causes the device 200 to perform at least some of the functionalities of the device 200.

In an embodiment, the sensor unit 210 comprises a circuitry including at least one processor and at least one memory including computer program code, wherein the circuitry causes the sensor unit 210 to perform at least some of the functionalities of the sensor unit 210.

As used in this application, the term ‘circuitry’ refers to all of the following: (a) hardware-only circuit implementations, such as implementations in only analog and/or digital circuitry, and (b) combinations of circuits and software (and/or firmware), such as (as applicable): (i) a combination of processor(s) or (ii) portions of processor(s)/software including digital signal processor(s), software, and memory(ies) that work together to cause an apparatus to perform various functions, and (c) circuits, such as a microprocessor(s) or a portion of a microprocessor(s), that require software or firmware for operation, even if the software or firmware is not physically present. This definition of ‘circuitry’ applies to all uses of this term in this application. As a further example, as used in this application, the term ‘circuitry’ would also cover an implementation of merely a processor (or multiple processors) or a portion of a processor and its (or their) accompanying software and/or firmware.

In an embodiment, at least some of the functionalities according to any one of the embodiments or operations thereof may be carried out by an apparatus comprising corresponding means for carrying out at least some of the described processes. Some example means for carrying out the processes may include at least one of the following: detector, processor (including dual-core and multiple-core processors), digital signal processor, controller, receiver, transmitter, encoder, decoder, memory, RAM, ROM, software, firmware, display, user interface, display circuitry, user interface circuitry, user interface software, display software, circuit, antenna, antenna circuitry, and circuitry. In an embodiment, the at least one processor, the memory, and the computer program code form processing means or comprises one or more computer program code portions for carrying out one or more operations according to any one of the embodiments or operations thereof.

The techniques and methods described herein may be implemented by various means. For example, these techniques may be implemented in hardware (one or more devices), firmware (one or more devices), software (one or more modules), or combinations thereof. For a hardware implementation, the apparatus(es) of embodiments may be implemented within one or more application-specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described herein, or a combination thereof. For firmware or software, the implementation can be carried out through modules of at least one chip set (e.g. procedures, functions, and so on) that perform the functions described herein. The software codes may be stored in a memory unit and executed by processors. The memory unit may be implemented within the processor or externally to the processor. In the latter case, it can be communicatively coupled to the processor via various means, as is known in the art. Additionally, the components of the systems described herein may be rearranged and/or complemented by additional components in order to facilitate the achievements of the various aspects, described with regard thereto, and they are not limited to the precise configurations set forth in the given figures, as will be appreciated by one skilled in the art.

Embodiments as described may also be carried out in the form of a computer process defined by a computer program. The computer program may be in source code form, object code form, or in some intermediate form, and it may be stored in some sort of carrier, which may be any entity or device capable of carrying the program. For example, the computer program may be stored on a computer program distribution medium readable by a computer or a processor. The distribution medium may be non-transitory and/or transitory, for example. The computer program medium may be, for example but not limited to, a record medium, computer memory, read-only memory, electrical carrier signal, telecommunications signal, and software distribution package, for example. Coding of software for carrying out the embodiments as shown and described is well within the scope of a person of ordinary skill in the art.

Even though the invention has been described above with reference to an example according to the accompanying drawings, it is clear that the invention is not restricted thereto but can be modified in several ways within the scope of the appended claims. Therefore, all words and expressions should be interpreted broadly and they are intended to illustrate, not to restrict, the embodiment. It will be obvious to a person skilled in the art that, as technology advances, the inventive concept can be implemented in various ways. Further, it is clear to a person skilled in the art that the described embodiments may, but are not required to, be combined with other embodiments in various ways.

1. A device for measuring physical activity of a user during swimming, the device comprising:
a sensor unit adapted and dimensioned to be coupled with a swimming goggles, the sensor unit comprising a motion circuitry configured to measure physical motion of the user, an optical cardiac activity circuitry configured to be placed at least partially against a body tissue of the user and to measure cardiac activity of the user, and a wireless communication circuitry configured to transfer data between the device and at least one external device,
wherein the wireless communication circuitry is configured to transfer physical activity data of the user to the at least one external device, the physical activity data comprising at least one of motion data obtained using the motion circuitry, cardiac activity data obtained using the cardiac activity circuitry,
the sensor unit comprising:
an interface element and a controller, the controller configured to change an operating mode of the sensor unit such that when the interface element is electrically coupled with a corresponding interface element of an audio module, the sensor unit operates in a control mode in which the sensor unit controls the audio module to output sound,
and such that when the interface element is electrically coupled with a corresponding interface element of an external device providing a power input, the sensor unit operates in a maintenance mode in which the sensor unit is recharged by said external device providing said power input;
wherein the audio module comprises an electroacoustic transducer configured to be placed against the body tissue of the user and to convert electrical signals into mechanical vibrations to produce sound and/or to detect sound as mechanical vibrations and to covert the mechanical vibrations into electrical signals;
wherein the device is configured to cause the audio module to output one or more sound notifications;
wherein the device is configured to detect a turn by the user during swimming and to cause the audio module to output a notification indicating a recorded time; and
wherein the device is further configured to determine a glide phase of the turn and to cause the audio module to output a notification indicating the recorded time during the glide phase.

2. The device of claim 1, wherein the wireless communication circuitry comprises a Bluetooth circuitry.

3. The device of claim 1, wherein the wireless communication circuitry is configured to operate on a 5 kHz radio band.

4. The device of claim 1, wherein the electroacoustic transducer comprises a bone conductor transducer.

5. The device of claim 1, wherein the device comprises the audio module wherein an interface element of the audio module is electrically coupled with the interface element of the sensor unit.

6. The device of claim 1, wherein the device is configured to cause the audio module to output at least one of a notification indicating a heart rate zone of the user or a notification indicating a heart rate of the user.

7. The device of claim 1, wherein the recorded time comprises at least one of a lap time of the user, a pool length time of the user, a total swimming time.

8. The device of claim 1, wherein the detecting the turn is based on at least one of the motion data obtained using the motion circuitry, data obtained using one or more external sensor devices worn by the user.

9. The device of claim 1, wherein the sensor unit is detachably attachable to the audio module.

10. The device of claim 4, wherein the audio module comprises at least one attachment element for attaching the device to a strap of the swimming goggles.

11. The device of claim 1, wherein the sensor unit comprises a power source, and wherein the interface element and the power source are configured to provide operational voltage to the audio module.

12. The device of claim 11, wherein the operational voltage to the audio module is transferred via the interface element as alternating current voltage.

13. The device of claim 1, wherein in the maintenance mode the sensor unit is further configured to transmit physical activity data to said external device, and/or to receive configuration data from said external device.

14. The device of claim 1, wherein the device is configured to determine, based on the motion data and/or measurement by the wireless communication circuitry, at least one time period when the device is above water during swimming, and wherein the device is further configured to transfer the physical activity data of the user to the at least one external device during said at least one time period.

15. The device of claim 1, wherein the device is configured to determine that the device is used during swimming, and wherein the device is further configured to decrease transmission interval of the wireless communication circuitry during swimming.

16. The device of claim 1, further comprising:
an electromagnetic flowmeter configured to measure swimming speed of the user, wherein the physical activity data comprises the swimming speed and/or the device is further configured to cause the audio module to output a notification indicating the swimming speed.

17. The device of claim 1, wherein said interface element and the corresponding interface element of the audio module enable at least one of communication between the sensor unit and the audio module, transfer of operational voltage from the sensor unit to the audio module.

18. The device of claim 1, further comprising:
one or more light emitting diodes configured to output visual notifications to the user.

19. The device of claim 18, further comprising:
one or more light guides configured to convey light emitted by the one or more light emitting diodes to one or more lens areas of the swimming goggles.
